(19) United States
(12) Patent Application Publication
(10) Pub. No.: US 2021/0317436 A1
(43) Pub. Date: Oct. 14, 2021

(54) METHODS AND COMPOSITIONS FOR MODIFYING THE VON WILLEBRAND FACTOR GENE

(71) Applicant: BLUEALLELE, LLC, Maple Grove, MN (US)

(72) Inventor: Nicholas BALTES, Maple Grove, MN (US)

(21) Appl. No.: 17/273,720

(22) PCT Filed: Sep. 6, 2019
(86) PCT No.: PCT/US2019/049850

§ 371 Date: Mar. 4, 2021

Related U.S. Application Data
(60) Provisional application No. 62/728,760, filed on Sep. 8, 2018.

(51) Int. Cl. C12N 15/10; C12N 9/22; C12N 15/86; C12N 15/90

(52) U.S. Cl. CPC C12N 15/102; C12N 9/22; C07K 14/745; C12N 15/907; C12N 2750/14143; C12N 15/86

(57) ABSTRACT

Methods and compositions for modifying the coding sequence of endogenous genes using rare-cutting endonucleases. The methods and compositions described herein can be used to modify the endogenous von Willebrand factor gene.

### REFERENCE TO RELATED APPLICATION

This application claims priority to previously filed and co-pending provisional application U.S. Ser. No. 62/728,760, FILED Sep. 8, 2018, the contents of which are incorporated herein by reference.

### SEQUENCE LISTING

The instant application contains a Sequence Listing which has been submitted in ASCII format via EFS-Web and is hereby incorporated by reference in its entirety. Said ASCII copy, created on Sep. 4, 2019 is named BA2018-2PRIO SEQUENCE LISTING and is 107,084 bytes in size.

### TECHNICAL FIELD

The present document is in the field of gene therapy and genome editing. More specifically, this document relates to the targeted modification of endogenous genes, including the von Willebrand factor gene for treatment of genetic disorders.

### BACKGROUND

Monogenic disorders are caused by one or more mutations in a single gene, examples of which include sickle cell disease (hemoglobin-beta gene), cystic fibrosis (cystic fibrosis transmembrane conductance regulator gene), and Tay-Sachs disease (beta-hexosaminidase A gene). Monogenic disorders have been an interest for gene therapy, as replacement of the defective gene with a functional copy could provide therapeutic benefits. However, one bottleneck for generating effective therapies includes the size of the functional copy of the gene. Many delivery methods, including those that use viruses, have size limitations which hinder the delivery of large transgenes. Methods to correct partial regions of a defective gene may provide an alternative means to treat monogenic disorders.

Von Willebrand disease (vWD) is a monogenic disorder and is reported to be the most common inherited bleeding disorder in humans and is caused by quantitative or qualitative defects in the von Willebrand factor (vWF) protein. vWF is a glycoprotein within plasma and is present as a series of multimers ranging in size from about 500 to 20,000 kD. Multimeric forms of vWF are composed of 250 kD polypeptide subunits linked together by disulfide bonds. vWF mediates the initial platelet adhesion to the subendothelium of a damaged vessel wall. In addition, vWF protects factor (F) VIII from proteolytic degradation by binding to and transporting FVIII to the site of coagulation. Expression of the vWF gene is primarily in vascular endothelial cells and megakaryocytes.

vWD is classified into three categories: type 1, type 2 and type 3. Based on properties of the vWF protein, type 2 can be further classified as 2A, 2B, 2M and 2N. The categories general define the quantitative or qualitative deficiencies of the vWF protein: type 1 relates to the partial quantitative deficiency of vWF and an associated decrease in FVIII levels; type 2A relates to defective vWF-platelet binding properties and decreased high molecular weight multimers; type 2B relates to increased vWF-platelet Gp1b binding and decreased high molecular weight multimers; type 2M relates to defective vWF-platelet binding and dysfunctional high molecular weight multimers; type 2N relates to a lack or reduction in vWF affinity for FVIII binding; type 3 relates to a complete deficiency of vWF and severely reduced FVIII levels.

Current treatment strategies for vWD are based on enzyme replacement of the defective vWF protein. Although protein replacement therapy or desmopressin-induced vWF release is adequate for the majority of patients, only a short-term effect can be achieved due to the short half-life of vWF. Therefore, there is increasing interest to develop gene therapies for extended vWF production.

The vWF gene is located on the short arm of chromosome 12 at position 13.31 and the genomic sequence spans 178-kb and comprises 52 exons. Exon 28 is the largest at 1,379 bp long. Since vWD is a monogenic disease it is a good candidate for gene therapy; however, for gene therapy using virus vectors such as those based upon adeno-associated virus, the coding sequence (˜8.4 kb) is too large to fit into a single vector.

Development of methods and materials for correcting defective vWF genes could provide additional therapeutic options for those with vWD.

### SUMMARY

Gene editing holds promise for correcting mutations found in genetic disorders; however, many challenges remain for creating effective therapies for individual disorders, including those that are caused by mutations present throughout relatively large genes, or disorders where the gene is primarily expressed in tissue that common delivery tools have difficulty accessing. These challenges are seen with disorders such as the blood clotting disorder, von Willebrand disease. The von Willebrand factor is a stored within the Weibel-Palade bodies (WPBs) of endothelial cells as a highly prothrombotic protein and is release under tight control. The coding sequence is approximately 8.4 kb, which is too large to fit on most current delivery vehicles.

The methods described herein provide novel approaches for correcting mutations found in the vWF gene. The methods are compatible with current delivery vehicles (e.g. adeno-associated virus vectors and lipid nanoparticles), and they address the challenges due to the size, structure and expression of vWF. In one embodiment, a transgene can be integrated into the vWF gene for correcting mutations. The transgene can contain a partial coding sequence of the vWF gene. For example, exons 1-20 of the endogenous von Willebrand factor gene can be replaced with a partial synthetic von Willebrand coding sequence comprising sequence homologous to exons 2-20. Further, the modification can include integration of a promoter, enabling expression of the corrected von Willebrand gene in tissue that normally does not express vWF, including liver tissue. In another example, exons 29-52 of the endogenous von Willebrand factor gene can be replaced with a partial synthetic von Willebrand coding sequence comprising sequence homologous to exons 29-52. The methods described herein can be used to correct or introduce genetic modifications in endogenous genes. The modifications can be used for applied research (gene therapy) or basic research (creation of animal models or understanding gene function).

In one embodiment, this document features a method for integrating a transgene into the von Willebrand factor gene. The method can include transfecting a cell with a rare-cutting endonuclease or transposase which is targeted to the von Willebrand factor gene, along with transfecting a transgene. The transgene can integrate into the von Willebrand factor gene following cleavage by the rare-cutting endonuclease or integration by the transposase. The transgene can comprise sequence that is homologous to one or more exons within the von Willebrand factor gene. The cell being transfected can include a hepatic cell, an induced pluripotent stem cell (iPSC), a hematopoietic stem cell, a hepatic cell, a hepatic stem cell, or a red blood precursor cell. The cell can be transfected with a transgene comprising exons 2-20 (i.e., exons 2, 3, 4, 5, 6, 7, 8, 9, 10, 11, 12, 13, 14, 15, 16, 17, 18, 19 and 20) of the von Willebrand factor gene. The transgene can comprise a promoter driving expression of the partial coding sequence. In another embodiment, the cell being transfected can be an endothelial cell. The endothelial cell can be transfected with a transgene comprising exons 29-52 of the von Willebrand factor gene. The exons can be operably linked to a terminator. The transgenes, either containing the promoter or terminator, can be integrated within an intron within an endogenous von Willebrand factor gene. The rare-cutting endonucleases, which facilitate the integration of the transgene, can include a zinc-finger nuclease, a transcription activator-like effector nuclease, or a CRISPR/Cas endonuclease. The transgene can be delivered to cells using viral vectors, including adenoviral (Ad) vectors or an adeno-associated viral (AAV) vectors. The transposase which facilitates integration of the transgene can include CRISPR-associated transposase systems. These systems can include Cas12k or Cas6.

In another embodiment, this document provides a method of modifying genomic DNA, where the method includes administering a rare-cutting endonuclease or transposase targeted to a site within the von Willebrand factor gene in a hepatocyte or endothelial cell, and administering a transgene, wherein the transgene is integrated within the von Willebrand factor gene. The method can include the use of a CRISPR-associated transposase, including those having Cas12k or Cas6. The Cas12k sequence can be from Scytonema hofmanni or Anabaena cylindrica. The rare-cutting endonuclease can be selected from a CRISPR nuclease, TAL effector nuclease, zinc-finger nuclease, or meganuclease. The target von Willebrand factor gene can include a gene with one or more mutations that cause von Willebrand disease (i.e., vWD Type 1, 2 or 3).

The methods described herein can also be extended to genes associated with other genetic disorders. As described herein, the other genes can include the IDS gene (Hunter Syndrome), GLA gene (Fabry disease), GAA gene (Pompe disease), ARSB gene (Maroteaux-Lamy syndrome), GALNS gene (Morquio A syndrome), GLB1 gene (Morquio A syndrome), LIPA gene (Lysosomal acid lipase deficiency), F8 gene (Hemophilia A), F9 gene (Hemophilia B), and F11 gene (Hemophilia C). The modification can include the N′ terminus of the endogenous protein through integrating a promoter, partial coding sequence and splice donor into the endogenous gene. The modification can occur in hepatocytes.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the description below. Other features, objects, and advantages of the invention will be apparent from the description and from the claims.

### DESCRIPTION OF DRAWINGS

FIG. 1 is an illustration of the human von Willebrand factor genomic sequence. Shown is the genomic region comprising exons 20-28 and potential target sites for transgene comprising vWF coding sequence (cDNA).

FIG. 2 is an illustration of an adeno-associated vector comprising exons 2-20 of the von Willebrand factor gene.

FIG. 3 is an illustration of the method to integrate a transgene comprising a promoter operably linked to exons 2-20 of the von Willebrand factor gene into the endogenous von Willebrand factor gene. Also shown is the transcriptional product that is generated after integration occurs.

FIG. 4 is an illustration of the human von Willebrand factor genomic sequence. Shown is the genomic region comprising exons 28-35 and potential target sites for transgene comprising vWF coding sequence (cDNA).

FIG. 5 is an illustration of an adeno-associated vector comprising exons 29-52 of the von Willebrand factor gene.

FIG. 6 is an illustration of the method to integrate a transgene comprising a terminator operably linked to exons 29-52 of the von Willebrand factor gene into the endogenous von Willebrand factor gene. Also shown is the transcriptional product that is generated after integration occurs.

FIG. 7 is an illustration of the integration of a transgene comprising the hCMV-intron promoter upstream of exons 2-20. Also shown is the location of primers for analyzing the integration event.

FIG. 8 is an image of gels detecting integration of partial vWF coding sequences within the vWF gene.

FIG. 9 is a graph showing the expression levels of modified vWF genes normalized to an internal control (GAPDH).

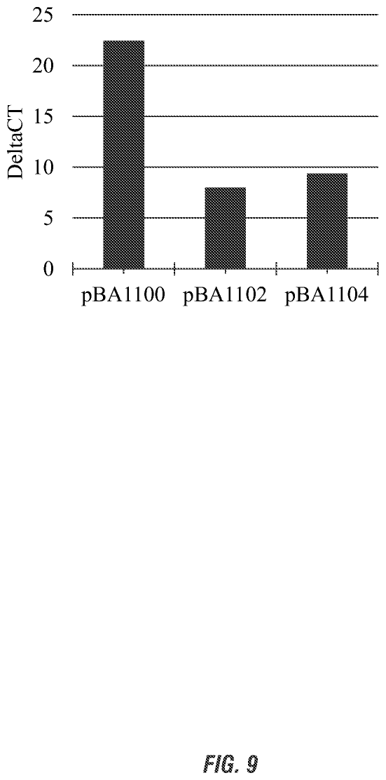

### DETAILED DESCRIPTION

Disclosed herein are methods and compositions for modifying the coding sequence of endogenous genes. In some embodiments, the methods include inserting a transgene into an endogenous gene, wherein the transgene provides a partial coding sequence which substitutes for the endogenous gene's coding sequence.

In one embodiment, this document provides a method of integrating a transgene into the von Willebrand factor gene, where the method comprises administering a rare-cutting endonuclease or transposase targeted to a site within the von Willebrand factor gene, and administering a transgene, wherein the transgene is integrated within the von Willebrand factor gene. The method can include the use of a CRISPR-associated transposase, including those having Cas12k or Cas6. The Cas12k sequence can be from Scytonema hofmanni or Anabaena cylindrica. The rare-cutting endonuclease can be selected from a CRISPR nuclease, TAL effector nuclease, zinc-finger nuclease, or meganuclease. The target von Willebrand factor gene can include a gene with one or more mutations that cause von Willebrand disease (i.e., vWD Type 1, 2 or 3). In one aspect, the target von Willebrand factor gene comprises mutations that cause Type 2N or Type 3 vWD. The transgene integrated into the vWF gene can include a promoter, a partial vWF coding sequence from a functional vWF gene, and a splice donor. Specifically, the partial coding sequence can comprise vWF exons 2-20, or it can encode for the peptide produced by exons 2-20 of a functional vWF gene. This transgene can be integrated in exon 20 or intron 20 of the aberrant vWF gene. In another embodiment, the partial coding sequence comprises vWF exons 2-22, or encodes for the peptide produced by exons 2-22 of a functional vWF gene. Here, the transgene can be integrated in exon 22 or intron 22 of the vWF gene. In another embodiment, the partial coding sequence comprises vWF exons 2-27, or encodes for the peptide produced by exons 2-27 of a functional vWF gene. Here, the transgene is integrated in exon 27 or intron 27 of the vWF gene. In another embodiment, the transgene for integration into vWF can comprise a splice acceptor, a partial vWF coding sequence from a functional vWF gene, and a terminator. The partial coding sequence can comprise vWF exons 35-52, or encodes for the peptide produced by exons 35-52 of a functional vWF gene. Here, the transgene can be integrated in intron 34 of the vWF gene. In another embodiment, the partial coding sequence comprises vWF exons 33-52, or encodes for the peptide produced by exons 33-52 of a functional vWF gene. Here, the transgene is integrated in intron 32 of the vWF gene. In another embodiment, the partial coding sequence comprises vWF exons 29-52, or encodes for the peptide produced by exons 29-52 of a functional vWF gene. Here, the transgene is integrated in intron 28 of the vWF gene. In all variations of the transgene, the transgene can be integrated through HR, NHEJ or transposition. If integrated by transposition, the transgene can comprise left and right ends compatible with a corresponding transposase. If integrated by HR, the transgene can comprise a left and right homology arm. Regarding transgenes comprising a promoter and partial coding sequence and splice donor, the transgene can be administered to a cell, and the cell can be selected from a hepatocyte, an induced pluripotent stem cell (iPSC), a hematopoietic stem cell, a hepatic cell, a hepatic stem cell, or a red blood precursor cell. Specifically, the cell can be a hepatocyte. Regarding transgenes comprising a terminator, partial coding sequence and splice acceptor, the transgene can be administered to an endothelial cell. When administering the transgene to a cell, the transgene can be harbored on an adeno-associated virus vector. In another embodiment, the transgene can be administered together with lipid nanoparticles. The promoter present on the transgene comprising a promoter and partial coding sequence and splice donor can be a tissue specific promoter, inducible promoter, or constitutive promoter. Specifically, the promoter can be an inducible promoter.

In another embodiment, this document provides a method of modifying genomic DNA, where the method includes administering a rare-cutting endonuclease or transposase targeted to a site within the von Willebrand factor gene in a hepatocyte or endothelial cell, and administering a transgene, wherein the transgene is integrated within the von Willebrand factor gene. The method can include the use of a CRISPR-associated transposase, including those having Cas12k or Cas6. The Cas12k sequence can be from Scytonema hofmanni or Anabaena cylindrica. The rare-cutting endonuclease can be selected from a CRISPR nuclease, TAL effector nuclease, zinc-finger nuclease, or meganuclease. The target von Willebrand factor gene can include a gene with one or more mutations that cause von Willebrand disease (i.e., vWD Type 1, 2 or 3). In one aspect, the target von Willebrand factor gene comprises mutations that cause Type 2N or Type 3 vWD. The transgene integrated into the vWF gene can include a promoter, a partial vWF coding sequence from a functional vWF gene, and a splice donor. Specifically, the partial coding sequence can comprise vWF exons 2-20, or it can encode for the peptide produced by exons 2-20 of a functional vWF gene. This transgene can be integrated in exon 20 or intron 20 of the aberrant vWF gene. In another embodiment, the partial coding sequence comprises vWF exons 2-22, or encodes for the peptide produced by exons 2-22 of a functional vWF gene. Here, the transgene can be integrated in exon 22 or intron 22 of the vWF gene. In another embodiment, the partial coding sequence comprises vWF exons 2-27, or encodes for the peptide produced by exons 2-27 of a functional vWF gene. Here, the transgene is integrated in exon 27 or intron 27 of the vWF gene. In another embodiment, the transgene for integration into vWF can comprise a splice acceptor, a partial vWF coding sequence from a functional vWF gene, and a terminator. The partial coding sequence can comprise vWF exons 35-52, or encodes for the peptide produced by exons 35-52 of a functional vWF gene. Here, the transgene can be integrated in intron 34 of the vWF gene. In another embodiment, the partial coding sequence comprises vWF exons 33-52, or encodes for the peptide produced by exons 33-52 of a functional vWF gene. Here, the transgene is integrated in intron 32 of the vWF gene. In another embodiment, the partial coding sequence comprises vWF exons 29-52, or encodes for the peptide produced by exons 29-52 of a functional vWF gene. Here, the transgene is integrated in intron 28 of the vWF gene. In all variations of the transgene, the transgene can be integrated through HR, NHEJ or transposition. If integrated by transposition, the transgene can comprise left and right ends compatible with a corresponding transposase. If integrated by HR, the transgene can comprise a left and right homology arm. Regarding transgenes comprising a promoter and partial coding sequence and splice donor, the transgene can be administered to a cell, and the cell can be a hepatocyte. Regarding transgenes comprising a terminator, partial coding sequence and splice acceptor, the transgene can be administered to an endothelial cell. When administering the transgene to a cell, the transgene can be harbored on an adeno-associated virus vector. In another embodiment, the transgene can be administered together with lipid nanoparticles. The promoter present on the transgene comprising a promoter and partial coding sequence and splice donor can be a tissue specific promoter, inducible promoter, or constitutive promoter. Specifically, the promoter can be an inducible promoter.

In another embodiment, this document provides an isolated nucleic acid comprising a promoter, a partial coding sequence of a functional gene, a splice donor sequence, and a left and right homology arm or a transposon left end and right end. The nucleic acid can include a partial vWF coding sequence. The partial vWF coding sequence can include vWF exons 2-20, or the encode for the peptide produced by exons 2-20 of a functional vWF gene. In another embodiment, the nucleic acid can include vWF exons 2-22, or encode for the peptide produced by exons 2-22 of a functional vWF gene. In another embodiment, the nucleic acid can include vWF exons 2-27, or encode for the peptide produced by exons 2-27 of the wild type vWF gene. In an embodiment, the isolated nucleic acid sequence can contain a tissue specific promoter, inducible promoter, or constitutive promoter. Specifically, the promoter can be an inducible promoter.

In another embodiment, this document provides an isolated nucleic acid comprising a splice acceptor sequence, a partial coding sequence of a functional gene, a terminator, and a left and right homology arm or a transposon left end and right end. The nucleic acid can include a partial vWF coding sequence. The partial vWF coding sequence can include vWF exons 35-52, or encode for the peptide produced by exons 35-52 of a functional vWF gene. In another embodiment, the partial vWF coding sequence can include vWF exons 33-52, or encode for the peptide produced by exons 33-52 of a functional vWF gene. In another embodiment, the partial vWF coding sequence can include vWF exons 29-52, or encode for the peptide produced by exons 29-52 of a functional vWF gene.

In an embodiment, his document provides a method of altering expression of a gene in a cell, where the method includes administering a rare-cutting endonuclease or transposase targeted to a site within the gene, and administering a transgene, wherein the transgene is integrated within the gene and expression of the gene is increased as compared to expression of the gene from a wild type cell. The method can include the use of a CRISPR-associated transposase, including those having Cas12k or Cas6. The Cas12k sequence can be from Scytonema hofmanni or Anabaena cylindrica. The rare-cutting endonuclease can be selected from a CRISPR nuclease, TAL effector nuclease, zinc-finger nuclease, or meganuclease. The method can include the use of a transgene which comprises a promoter, a partial coding sequence, and a splice donor. The transgene can be integrated into a gene that is associated with a genetic disorder, including the IDS gene (Hunter Syndrome), GLA gene (Fabry disease), GAA gene (Pompe disease), ARSB gene (Maroteaux-Lamy syndrome), GALNS gene (Morquio A syndrome), GLB1 gene (Morquio A syndrome), LIPA gene (Lysosomal acid lipase deficiency), F8 gene (Hemophilia A), F9 gene (Hemophilia B), F11 gene (Hemophilia C), and vWF gene (Von Willebrand disease). The modification can include the N′ terminus of the endogenous protein through integrating a promoter, partial coding sequence and splice donor into the endogenous gene. The modification can occur in hepatocytes.

Practice of the methods, as well as preparation and use of the compositions disclosed herein employ, unless otherwise indicated, conventional techniques in molecular biology, biochemistry, chromatin structure and analysis, computational chemistry, cell culture, recombinant DNA and related fields as are within the skill of the art. These techniques are fully explained in the literature. See, for example, Sambrook et al. MOLECULAR CLONING: A LABORATORY MANUAL, Second edition, Cold Spring Harbor Laboratory Press, 1989 and Third edition, 2001; Ausubel et al., CURRENT PROTOCOLS IN MOLECULAR BIOLOGY, John Wiley & Sons, New York, 1987 and periodic updates; the series METHODS IN ENZYMOLOGY, Academic Press, San Diego; Wolffe, CHROMATIN STRUCTURE AND FUNCTION, Third edition, Academic Press, San Diego, 1998; METHODS IN ENZYMOLOGY, Vol. 304, “Chromatin” (P. M. Wassarman and A. P. Wolffe, eds.), Academic Press, San Diego, 1999; and METHODS IN MOLECULAR BIOLOGY, Vol. 119, “Chromatin Protocols” (P. B. Becker, ed.) Humana Press, Totowa, 1999.

As used herein, the terms “nucleic acid” and “polynucleotide,” can be used interchangeably. Nucleic acid and polynucleotide can refer to a deoxyribonucleotide or ribonucleotide polymer, in linear or circular conformation, and in either single- or double-stranded form. These terms are not to be construed as limiting with respect to the length of a polymer. The terms can encompass known analogues of natural nucleotides, as well as nucleotides that are modified in the base, sugar and/or phosphate moieties.

The terms “polypeptide,” “peptide” and “protein” can be used interchangeably to refer to amino acid residues covalently linked together. The term also applies to proteins in which one or more amino acids are chemical analogues or modified derivatives of corresponding naturally-occurring amino acids.

The terms “operatively linked” or “operably linked” are used interchangeably and refer to a juxtaposition of two or more components (such as sequence elements), in which the components are arranged such that both components function normally and allow the possibility that at least one of the components can mediate a function that is exerted upon at least one of the other components. By way of illustration, a transcriptional regulatory sequence, such as a promoter, is operatively linked to a coding sequence if the transcriptional regulatory sequence controls the level of transcription of the coding sequence in response to the presence or absence of one or more transcriptional regulatory factors. A transcriptional regulatory sequence is generally operatively linked in cis with a coding sequence, but need not be directly adjacent to it. For example, an enhancer is a transcriptional regulatory sequence that is operatively linked to a coding sequence, even though they are not contiguous.

As used herein, the term “cleavage” refers to the breakage of the covalent backbone of a nucleic acid molecule. Cleavage can be initiated by a variety of methods including, but not limited to, enzymatic or chemical hydrolysis of a phosphodiester bond. Cleavage can refer to both a single-stranded nick and a double-stranded break. A double-stranded break can occur as a result of two distinct single-stranded nicks. Nucleic acid cleavage can result in the production of either blunt ends or staggered ends. In certain embodiments, rare-cutting endonucleases are used for targeted double-stranded or single-stranded DNA cleavage.

An “exogenous” molecule can refer to a small molecule (e.g., sugars, lipids, amino acids, fatty acids, phenolic compounds, alkaloids), or a macromolecule (e.g., protein, nucleic acid, carbohydrate, lipid, glycoprotein, lipoprotein, polysaccharide), or any modified derivative of the above molecules, or any complex comprising one or more of the above molecules, generated or present outside of a cell, or not normally present in a cell. Exogenous molecules can be introduced into cells. Methods for the introduction of exogenous molecules into cells can include lipid-mediated transfer, electroporation, direct injection, cell fusion, particle bombardment, calcium phosphate co-precipitation, DEAE-dextran-mediated transfer and viral vector-mediated transfer.

An “endogenous” molecule is a small molecule or macromolecule that is present in a particular cell at a particular developmental stage under particular environmental conditions. An endogenous molecule can be a nucleic acid, a chromosome, the genome of a mitochondrion, chloroplast or other organelle, or a naturally-occurring episomal nucleic acid. Additional endogenous molecules can include proteins, for example, transcription factors and enzymes.

As used herein, a “gene,” refers to a DNA region encoding that encodes a gene product, including all DNA regions which regulate the production of the gene product. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions. As used herein, a “wild type gene” refers to a form of the gene that is present at the highest frequency in a particular population.

An “endogenous gene” refers to a DNA region normally present in a particular cell that encodes a gene product as well as all DNA regions which regulate the production of the gene product.

“Gene expression” refers to the conversion of the information, contained in a gene, into a gene product. A gene product can be the direct transcriptional product of a gene. For example, the gene product can be, but not limited to, mRNA, tRNA, rRNA, antisense RNA, ribozyme, structural RNA, or a protein produced by translation of an mRNA. Gene products also include RNAs which are modified, by processes such as capping, polyadenylation, methylation, and editing, and proteins modified by, for example, methylation, acetylation, phosphorylation, ubiquitination, ADP-ribosylation, myristilation, and glycosylation.

“Encoding” refers to the conversion of the information contained in a nucleic acid, into a product, wherein the product can result from the direct transcriptional product of a nucleic acid sequence. For example, the product can be, but not limited to, mRNA, tRNA, rRNA, antisense RNA, ribozyme, structural RNA, or a protein produced by translation of an mRNA. Gene products also include RNAs which are modified, by processes such as capping, polyadenylation, methylation, and editing, and proteins modified by, for example, methylation, acetylation, phosphorylation, ubiquitination, ADP-ribosylation, myristilation, and glycosylation.

As used herein, the term “recombination” refers to a process of exchange of genetic information between two polynucleotides. The term “homologous recombination (HR)” refers to a specialized form of recombination that can take place, for example, during the repair of double-strand breaks. Homologous recombination requires nucleotide sequence homology present on a “donor” molecule. The donor molecule can be used by the cell as a template for repair of a double-strand break. Information within the donor molecule that differs from the genomic sequence at or near the double-strand break can be stably incorporated into the cell's genomic DNA.

The term “homologous” as used herein refers to a sequence of nucleic acids or amino acids having similarity to a second sequence of nucleic acids or amino acids. In some embodiments, the homologous sequences can have at least 80% sequence identity (e.g., 81%, 85%, 90%, 95%, 96%, 97%, 98%, or 99% sequence identity) to one another.

The term “integrating” as used herein refers to the process of adding DNA to a target region of DNA. As described herein, integration can be facilitated by several different means, including non-homologous end joining, homologous recombination, or targeted transposition. By way of example, integration of a user-supplied DNA molecule into a target gene can be facilitated by non-homologous end joining. Here, a targeted-double strand break is made within the target gene and a user-supplied DNA molecule is administered. The user-supplied DNA molecule can comprise exposed DNA ends to facilitate capture during repair of the target gene by non-homologous end joining. The exposed ends can be present on the DNA molecule upon administration (i.e., administration of a linear DNA molecule) or created upon administration to the cell (i.e., a rare-cutting endonuclease cleaves the user-supplied DNA molecule within the cell to expose the ends). In another example, integration occurs though homologous recombination. Here, the user-supplied DNA harbors a left and right homology arm. In another example, integration occurs through transposition. Here, the user-supplied DNA harbors a transposon left and right end.

The term “transgene” as used herein refers to a sequence of nucleic acids that can be transferred to an organism or cell. The transgene may comprise a gene or sequence of nucleic acids not normally present in the target organism or cell. Additionally, the transgene may comprise a gene or sequence of nucleic acids that is normally present in the target organism or cell. A transgene can be an exogenous DNA sequence introduced into the cytoplasm or nucleus of a target cell. In one embodiment, the transgenes described herein contain a partial coding sequence, wherein the partial coding sequence encodes a portion of a protein that is functional, compared to that portion of the protein produced in the host.

The term “target gene” as used herein refers to an endogenous gene that is the target for modification. Further, the target gene can be present in two general forms: a “functional” gene or an “aberrant” gene. A functional target gene refers to gene that comprises a sequence of DNA which has the potential, under appropriate conditions, to encode a functional protein. Further, a functional gene refers to a gene that does not comprise a mutation associated or linked with a corresponding genetic disorder. By way of example, a wild type vWF gene is considered herein as a functional vWF gene. On the other hand, an aberrant gene refers to a gene that comprises mutations associated with or linked to a corresponding genetic disorder. The aberrant gene can encode an aberrant protein or can express a protein at reduced levels, as compared to a functional gene. The aberrant protein can be an inactive protein, a protein with reduced activity, or a protein with a gain-of-function mutation. By way of example, a functional vWF gene can encode a functional vWF protein as shown in SEQ ID NO:48. Additionally, a functional vWF gene can encode a functional variant of the vWF protein as shown in SEQ ID NO:48, so long as the variations are not associated with or linked to a corresponding genetic disorder (i.e., von Willebrand disease). Further, a functional vWF gene can be found in cells that do not primarily express the vWF protein (e.g., hepatocytes) so long as the gene does not comprise a mutation that is associated with or linked to a genetic disorder. On the other hand, an aberrant vWF gene can comprise loss-of-function or gain-of-function mutations which lead to phenotype associated with a genetic disorder. Aberrant vWF genes can include those found in patients with type 1, type 2 and type 3 von Willebrand disease. Specific examples of aberrant vWF genes include genes that are described in Freitas et al., Haemophilia 25:e78-85, 2019, Yadegari et al., Thrombosis and haemostasis 108:662-671, 2019, and Goodeve ASH Education Program Book 1:678-692, 2016, which are incorporated herein by reference.

The term “partial coding sequence” as used herein refers to a sequence of nucleic acids that encodes a partial protein. The partial coding sequence can encode a protein that comprises one or less amino acids as compared to the wild type protein or functional protein. The partial coding sequence can encode a partial protein with homology to the wild type protein or functional protein. The term “partial vWF coding sequence” as used herein refers to a sequence of nucleic acids that encodes a partial vWF protein. The partial vWF protein has one or less amino acids compared to a wild type vWF protein. The one or less amino acids can be from the N- or C-terminus end of the protein. If the partial vWF coding sequence is designed to amend the 5′ end of the vWF gene (i.e., the N-terminus of the vWF protein), then the partial vWF coding sequence can encode a minimum of the first 18 amino acids (i.e., the coding region of the first exon) of the vWF protein, and a maximum of first 2751 amino acids of the vWF protein. The first 18 amino acids can be the amino acids shown in SEQ ID NO:49. The first 2751 amino acids can be the amino acids shown in SEQ ID NO:50. If the partial vWF coding sequence is designed to amend the 3′ end of the vWF gene (i.e., the C-terminus of the vWF protein), then the partial vWF coding sequence can encode a minimum of the last 62 amino acids (i.e., the coding region in the last exon) of the vWF protein, and a maximum of last 2795 amino acids of the vWF protein. The last 62 amino acids can be the amino acids shown in SEQ ID NO:51. The last 2795 amino acids can be the amino acids shown in SEQ ID NO:52.

An embodiment provides for the transgene producing a functional fragment of the polypetide. A “functional fragment” of a protein, polypeptide or nucleic acid is a protein, polypeptide or nucleic acid whose sequence is not identical to the full-length protein, polypeptide or nucleic acid, yet retains the same function as the full-length protein, polypeptide or nucleic acid. A functional fragment can possess more, fewer, or the same number of residues as the corresponding native molecule, and/or can contain one or more amino acid or nucleotide substitutions. Methods for determining the function of a nucleic acid (e.g., coding function, ability to hybridize to another nucleic acid) are well-known in the art. Similarly, methods for determining protein function are well-known. For example, the DNA-binding function of a polypeptide can be determined, for example, by filter-binding, electrophoretic mobility-shift, or immunoprecipitation assays. DNA cleavage can be assayed by gel electrophoresis. The ability of a protein to interact with another protein can be determined, for example, by co-immunoprecipitation, two-hybrid assays or complementation, both genetic and biochemical. See, for example, Fields et al. (1989) Nature 340:245-246; U.S. Pat. No. 5,585,245 and PCT WO 98/44350.

The transgene can also include “functional variants” of the von Willebrand factor gene disclosed. Functional variants include, for example, sequences having one or more nucleotide substitutions, deletions or insertions and wherein the variant retains functional polypeptide. Functional variants can be created by any of a number of methods available to one skilled in the art, such as by site-directed mutagenesis, induced mutation, identified as allelic variants, cleaving through use of restriction enzymes, or the like. Examples of functional variants for vWF include those described in James et al., Blood 109:145-154, 2007 and Bellissimo et al., Blood 119:2135-2140, 2012. These include, but are not limited to, L129M, G131S, T346I, L363F, R436C, A488G, A594G, A631V, P653L, M740I, H817Q, A837D, R854Q, R924Q, G967D, Q1030R, T1034del, P1162L, V1229G, N1231T, A1327T, R1342C, Y1584C, P1725S, A1795V, V1959M, P2063S, R2185Q, R2287W, R2313H, R2384W, T2647M, T2666M, P2695R, and V2793A.

The term “transposase” as used herein refers to one or more proteins that facilitate the integration of a transposon. A transposase can include a CRISPR-associated transposase (Strecker et al., Science 10.1126/science.aax9181, 2019; Klompe et al., Nature, 10.1038/s41586-019-1323-z, 2019). The transposases can be used in combination with a transgene comprising a transposon left end and right end. The CRISPR transposases can include the TypeV-U5, C2C5 CRISPR protein, Cas12k, along with proteins tnsB, tnsC, and tniQ. In some embodiments, the Cas12k can be from Scytonema hofmanni (SEQ ID NO:21) or Anabaena cylindrica (SEQ ID NO:22). Alternatively, the CRISPR transposase can include the Cas6 protein, along with helper proteins including Cas7, Cas8 and TniQ.

The terms “left end” and “right end” as used herein refers to a sequence of nucleic acids present on a transposon, which facilitates integration by a transposase. By way of example, integration of DNA using ShCas12k can be facilitated through a left end (SEQ ID NO:23) and right end sequence (SEQ ID NO:24) flanking a cargo sequence.

As used herein, the term “lipid nanoparticle” refers to a transfer vehicle comprising one or more lipids. The term “lipid nanoparticle” also refers to particles having at least one dimension on the order of nanometers (e.g., 1-1,000 nm) which include one or more lipids. The one or more lipids can be cationic lipids, non-cationic lipids, or PEG-modified lipids. The lipid nanoparticles can be formulated to deliver one or more gene editing reagents to one or more target cells. Examples of suitable lipids include phosphatidylglycerol, phosphatidylcholine, phosphatidylserine, phosphatidylethanolamine, sphingolipids, cerebrosides, and gangliosides. Also contemplated is the use of polymers as transfer vehicles, whether alone or in combination with other transfer vehicles. Suitable polymers may include, for example, polyacrylates, polyalkycyanoacrylates, polylactide, polylactide-polyglycolide copolymers, polycaprolactones, dextran, albumin, gelatin, alginate, collagen, chitosan, cyclodextrins, dendrimers and polyethylenimine. In one embodiment, the transfer vehicle is selected based upon its ability to facilitate the transfection of a gene editing reagent to a target cell. In an embodiment, the gene editing reagents can be delivered with the lipid nanoparticle BAMEA-016B. The gene editing reagents can be in the form of RNA. For example, the gene editing reagents can be Cas9 mRNA and sgRNA combined with BAMEA-016B lipid nanoparticles.

The percent sequence identity between a particular nucleic acid or amino acid sequence and a sequence referenced by a particular sequence identification number is determined as follows. First, a nucleic acid or amino acid sequence is compared to the sequence set forth in a particular sequence identification number using the BLAST 2 Sequences (Bl2seq) program from the stand-alone version of BLASTZ containing BLASTN version 2.0.14 and BLASTP version 2.0.14. This stand-alone version of BLASTZ can be obtained online at fr.com/blast or at ncbi.nlm.nih.gov. Instructions explaining how to use the Bl2seq program can be found in the readme file accompanying BLASTZ. Bl2seq performs a comparison between two sequences using either the BLASTN or BLASTP algorithm. BLASTN is used to compare nucleic acid sequences, while BLASTP is used to compare amino acid sequences. To compare two nucleic acid sequences, the options are set as follows: −i is set to a file containing the first nucleic acid sequence to be compared (e.g., C:\seq1.txt); −j is set to a file containing the second nucleic acid sequence to be compared (e.g., C:\seq2.txt); −p is set to blastn; −o is set to any desired file name (e.g., C:\output.txt); −q is set to −1; −r is set to 2; and all other options are left at their default setting. For example, the following command can be used to generate an output file containing a comparison between two sequences: C:\Bl2seq −i c:\seq1.txt −j c:\seq2.txt −p blastn −o c:\output.txt −q-l-r2. To compare two amino acid sequences, the options of Bl2seq are set as follows: −i is set to a file containing the first amino acid sequence to be compared (e.g., C:\seq1.txt); −j is set to a file containing the second amino acid sequence to be compared (e.g., C:\seq2.txt); −p is set to blastp; −o is set to any desired file name (e.g., C:\output.txt); and all other options are left at their default setting. For example, the following command can be used to generate an output file containing a comparison between two amino acid sequences: C:\Bl2seq −i c:\seq1.txt −j c:\seq2.txt −p blastp −o c:\output.txt. If the two compared sequences share homology, then the designated output file will present those regions of homology as aligned sequences. If the two compared sequences do not share homology, then the designated output file will not present aligned sequences.

Once aligned, the number of matches is determined by counting the number of positions where an identical nucleotide or amino acid residue is presented in both sequences. The percent sequence identity is determined by dividing the number of matches either by the length of the sequence set forth in the identified sequence, or by an articulated length (e.g., 100 consecutive nucleotides or amino acid residues from a sequence set forth in an identified sequence), followed by multiplying the resulting value by 100. The percent sequence identity value is rounded to the nearest tenth. In one embodiment, the methods described herein include modifying an endogenous von Willebrand factor gene. The modification can be the insertion of a transgene in the endogenous von Willebrand factor gene. The transgene can include a partial coding sequence for the von Willebrand protein. The partial coding sequence can be homologous to coding sequence within a wild type von Willebrand factor gene, or a functional variant of the wild type von Willebrand factor gene, or a mutant of the wild type von Willebrand factor gene. In some embodiments, the transgene encoding the partial von Willebrand protein is inserted into the 5′ end of an endogenous von Willebrand factor gene (i.e., within exons or introns 1-27). The transgene within the 5′ end of the von Willebrand factor gene can harbor a promoter and a partial von Willebrand coding sequence that functions to replace the endogenous exons present upstream of the site of integration. In other embodiments, the transgene encoding the partial von Willebrand protein is inserted into the 3′ end of an endogenous von Willebrand factor gene (i.e., within exons or introns 28-52). The transgene within the 3′ end of the von Willebrand factor gene can harbor a terminator and a partial von Willebrand factor coding sequence that functions to replace the endogenous exons present downstream of the site of integration. The methods described herein can be used to modify regions of the coding sequence for endogenous genes, including the von Willebrand factor gene.

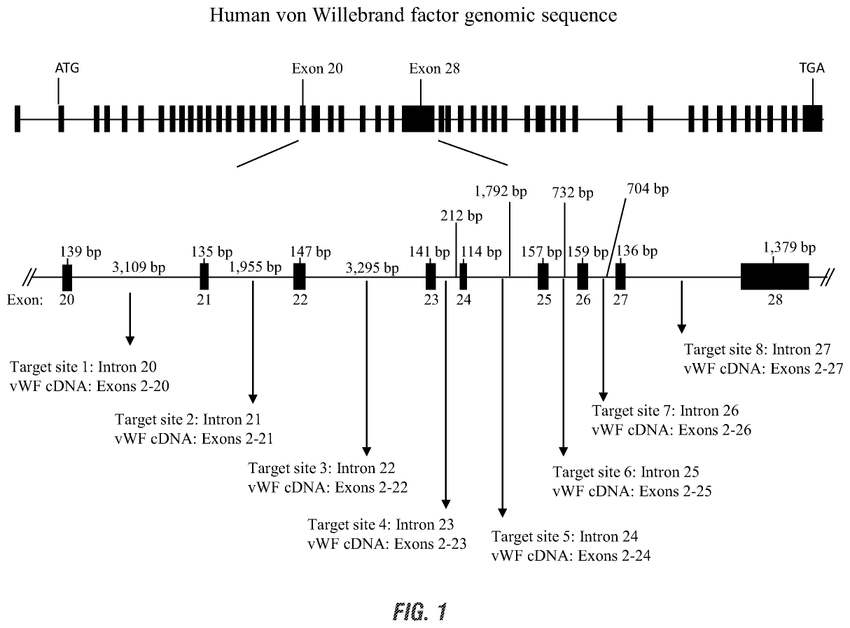

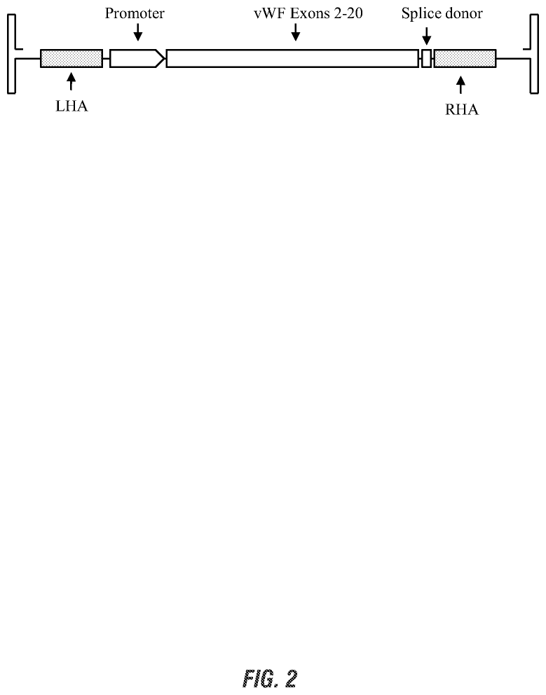

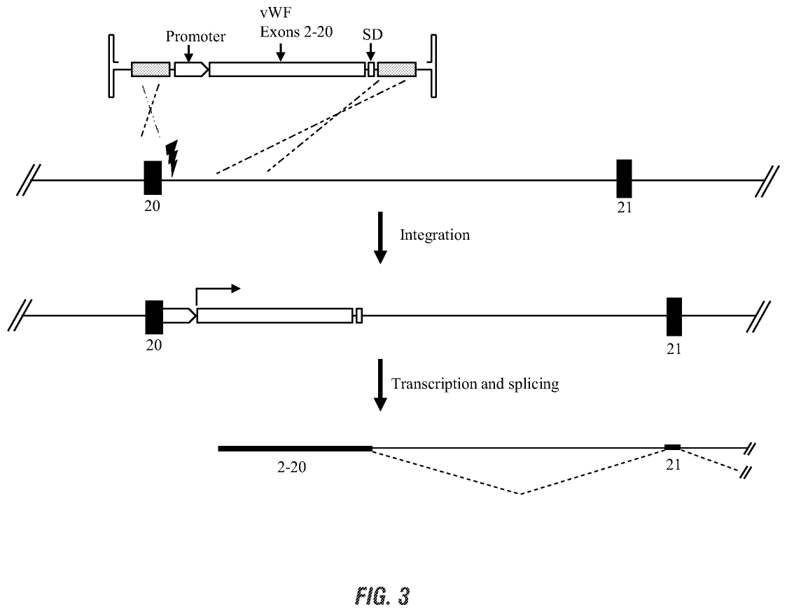

In one embodiment, the methods and compositions described herein can be used to modify the 5′ end of the vWF coding sequence, thereby resulting in modification of the N-terminus of the vWF protein (SEQ ID NO:48). As defined herein, modification of the 5′ end of the vWF coding sequence refers to the modification of at least the vWF exon comprising the start codon but not the exon comprising the stop codon. For example, the wild type vWF gene comprises 52 exons, with the stop codon being within exon 52. The modification of the 5′ end can include replacement of exons 1-51 of the vWF gene by a synthetic coding sequence. In other embodiments, the modification of the 5′ end of the vWF coding sequence can include the replacement of exons 1-27, or 2-27, or 2-26, or 2-25, or 2-24, or 2-23, or 2-22, or 2-21, or 2-20, or 2-19, or 2-18, or 2-17, or 2-16, or 2-15, or 2-14, or 2-13, or 2-12, or 2- 11, or 2-10, or 2-9, or 2-8, or 2-7, or 2-6, or 2-5, or 2-4, or 2-3. In one embodiment, the method to modify the 5′ end of the vWF coding sequence includes the integration of a transgene into the endogenous vWF gene. The transgene can harbor a partial synthetic vWF coding sequence comprising exons 1-27, or 2-27, or 2-26, or 2-25, or 2-24, or 2-23, or 2-22, or 2-21, or 2-20, or 2-19, or 2-18, or 2-17, or 2-16, or 2-15, or 2-14, or 2-13, or 2-12, or 2-11, or 2-10, or 2-9, or 2-8, or 2-7, or 2-6, or 2-5, or 2-4, or 2-3. The transgene harboring the partial synthetic vWF coding sequence can be integrated within the endogenous vWF gene at a site that is within or downstream of the exon which corresponds to the last exon of the partial synthetic coding sequence (FIG. 1). The synthetic vWF coding sequence can also comprise a promoter operably linked to the synthetic vWF coding sequence. The synthetic vWF coding sequence can also comprise a splice donor sequence which facilitates the splicing of the intron between the last exon within the synthetic vWF coding sequence and the downstream exon within the endogenous vWF sequence (FIGS. 2 and 3). The transgene can be designed in a donor molecule with arms of homology to a target site. Alternatively, the transgene can be designed in a transposon with left and right ends. The donor molecule or transposon can be incorporated into an AAV vector and particle and delivered in vivo to target cells. The target cells can comprise a vWF gene with either low or high gene expression. The target cells can be, for example, hepatocytes within the liver. The AAV comprising the donor molecule can be delivered with or without a second AAV encoding a rare-cutting endonuclease. The second AAV encoding a rare-cutting endonuclease can be used to facilitate recombination of the donor molecule with the endogenous vWF gene.

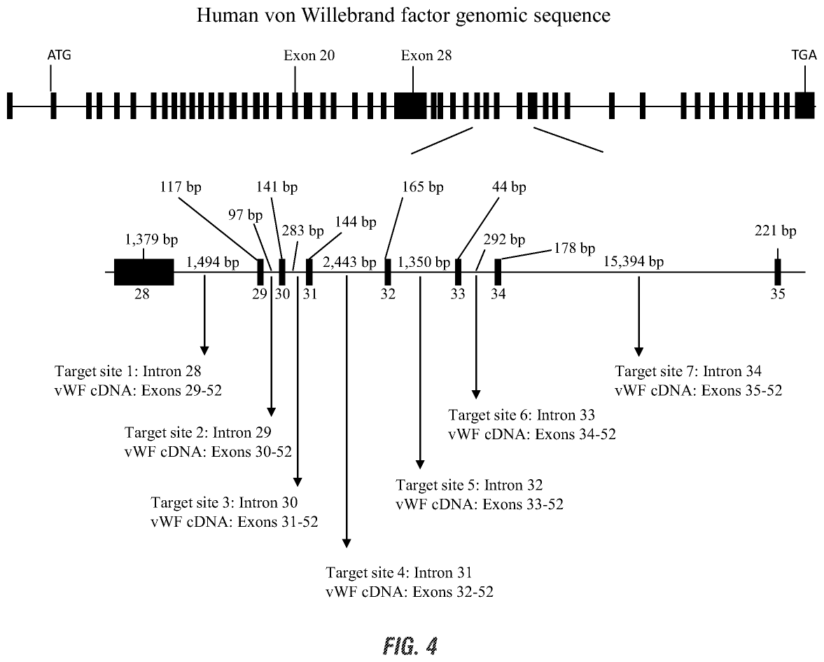

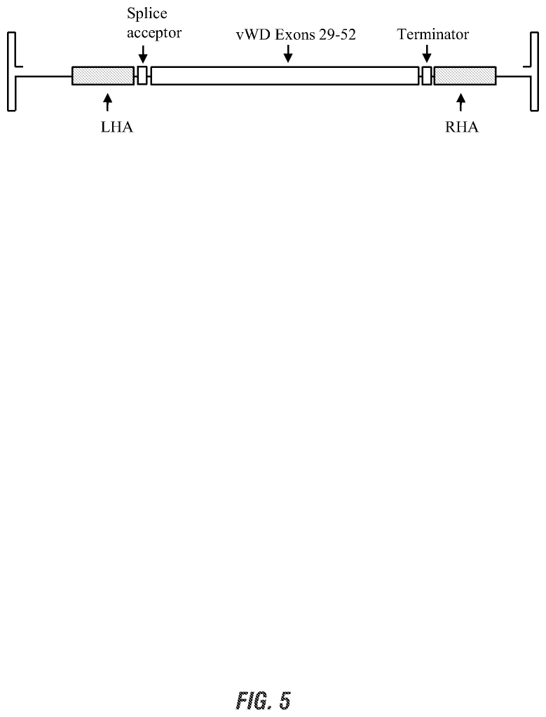

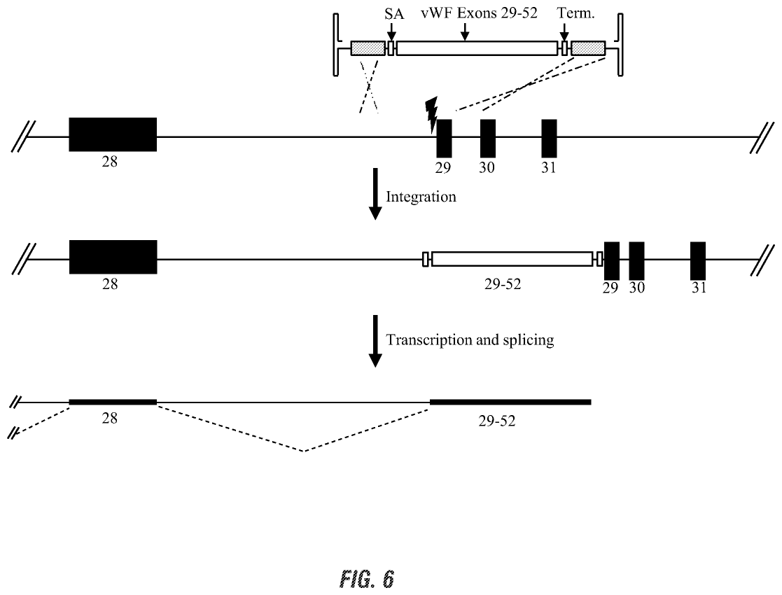

In another embodiment, the methods and compositions described herein can be used to modify the 3′ end of the vWF coding sequence, thereby resulting in modification of the C-terminus of the vWF protein. As defined herein, modification of the 3′ end of the vWF coding sequence refers to the modification of at least the vWF exon comprising the stop codon, but not the exon comprising the start codon. For example, the wild type vWF gene comprises 52 exons, with the start codon being within exon 2. The modification of the 3′ end can include replacement of exons 3-52 of the vWF gene by a synthetic vWF coding sequence. In other embodiments, the modification of the 3′ end of the vWF coding sequence can include the replacement of exons 28-52, or 29-52, or 30-52, or 31-52, or 32-52, or 33-52, or 34-52, or 35-52, or 36-52, or 37-52, or 38-52, or 39-52, or 40-52, or 41-52, or 42-52, or 43-52, or 44-52, or 45-52, or 46-52, or 47-52, or 48-52, or 49-52, or 50-52, or 51-52. In one embodiment, the method to modify the 3′ end of the vWF coding sequence includes the integration of a transgene into the endogenous vWF gene. The transgene can harbor a partial synthetic vWF coding sequence comprising exons 28-52, or 29-52, or 30-52, or 31-52, or 32-52, or 33-52, or 34-52, or 35-52, or 36-52, or 37-52, or 38-52, or 39-52, or 40-52, or 41-52, or 42-52, or 43-52, or 44-52, or 45-52, or 46-52, or 47-52, or 48-52, or 49-52, or 50-52, or 51-52. The partial synthetic vWF coding sequence can be integrated within the endogenous vWF gene upstream or within the exon which corresponds to the first exon within the partial synthetic vWF coding sequence (FIG. 4). The synthetic vWF coding sequence can comprise a terminater linked to the last exon in the synthetic vWF coding sequence. The partial synthetic vWF coding sequence can also comprise a splice acceptor sequence which facilitates the splicing of the intron between the first exon within the synthetic vWF coding sequence and the upstream exon within the endogenous vWF sequence (FIGS. 5 and 6). The transgene can be designed in a donor molecule with arms of homology to the target sequence. Alternatively, the transgene can be designed in a transposon with left and right ends. The donor molecule or transposon can be incorporated into an AAV vector and particle, and delivered in vivo to target cells. The target cells can comprise an endogenous vWF gene with moderate to high expression. The target cells can be, for example, endothelial cells lining blood vessels. The AAV comprising the donor molecule can be delivered with or without a second AAV encoding a rare-cutting endonuclease. The second AAV encoding a rare-cutting endonuclease can be used to facilitate recombination of the donor molecule with the endogenous vWF gene.

In one embodiment, the methods described herein involve the integration of a promoter, partial vWF coding sequence, and splice donor sequence into the von Willebrand gene. In a specific embodiment, the modification can occur in the vWF gene in hepatocytes. The promoter within the transgene can be a constitutive promoter, tissue specific promoter, inducible promoter or the native vWF promoter. The constitutive promoter can be, but not limited to, a CMV promoter, an EF1a promoter, an SV40 promoter, a PGK1 promoter, a Ubc promoter, a human beta actin promoter, or a CAG promoter. The inducible promoter can be, but not limited to, the tetracycline-dependent regulatable promoters or steroid hormone receptor promoters, including the promoters for the progesterone receptor regulatory system. The inducible promoter can be based upon ecdysone-based inducible systems, progesterone-based inducible systems, estrogen-based inducible systems, CID—(chemical inducers of dimerization) based systems or IPTG-based inducible systems. In one embodiment, the transgene comprising an inducible promoter, partial vWF coding sequence and splice donor sequence is integrated within the endogenous vWF gene in hepatocytes. To enable expression of the modified vWF gene, the cells are also administered nucleic acid or proteins to complete the system (e.g., the chimeric regulator GLVP for progesterone-based inducible systems) and are exposed to the inducer (RU486).

In some embodiments, the partial vWF coding sequence within the transgene can have homology to the corresponding wild type vWF coding sequence. The partial vWF coding sequence can have 100% homology to the corresponding vWF coding sequence found in human cells. In other embodiments, the partial vWF coding sequence can have minimal sequence homology to the corresponding wild type vWF coding sequence found in human cells. The partial vWF coding sequence can encode a protein with homology to the protein produced by a wild type vWF gene, however, the partial vWF coding sequence can be codon optimized or altered to have reduced or minimal sequence homology to the corresponding wild type vWF sequence.

In other embodiments, the transgene for altering the vWF gene can include a promoter, 5′ untranslated region, a partial vWF coding sequence, and a splice donor sequence. The 5′ untranslated region can be the endogenous vWF 5′ untranslated region, a synthetic 5′ untranslated region, or a 5′ untranslated region from a gene other than the vWF gene.

In other embodiments, the transgene for altering the vWF gene can include a splice acceptor sequence, a partial vWF coding sequence, a 3′ untranslated region, and a terminator. The 3′ untranslated region can be the endogenous vWF 3′ untranslated region, a synthetic 3′ untranslated region, or a 3′ untranslated region from a gene other than the vWF gene.

In some embodiments, the transgene for altering the vWF gene can encode a partial coding sequence of a functional vWF protein, and the target gene can be an aberrant vWF gene. In some embodiments, the aberrant vWF gene is within a host having von Willebrand disease. In some embodiments, the insertion of the partial coding sequence results in production of a functional vWF protein and increased levels of expression of the functional vWF protein.

In certain embodiments using the methods described herein, the level of polypeptide expression is increased by 2%, 3%, 4%, 5%, 6%, 7%, 8%, 9%, 10%, 20%, 25%, 30%, 35%, 40%, 45%, 50%, 55%, 60%, 65%, 70%, 80%, 85%, 90%, 95%, 100% or more or amounts in-between. In embodiments, the transgene encodes a partial functional protein, and upon successful integration, results in the expression of a functioning polypeptide that corrects defective vWF-platelet binding properties and decreased high molecular weight multimers; corrects increased vWF-platelet Gp1b binding and decreased high molecular weight multimers; corrects defective vWF-platelet binding and dysfunctional high molecular weight multimers; corrects a lack or reduction in vWF affinity for FVIII binding; and/or corrects complete deficiency of vWF and severely reduced FVIII levels.

In certain embodiments, the donor molecule can be in the form of circular or linear double-stranded or single stranded DNA. The donor molecule can be conjugated or associated with a reagent that facilitates stability or cellular update. The reagent can be lipids, calcium phosphate, cationic polymers, DEAE-dextran, dendrimers, polyethylene glycol (PEG) cell penetrating peptides, gas-encapsulated microbubbles or magnetic beads. The donor molecule can be incorporated into a viral particle. The virus can be retroviral, adenoviral, adeno-associated vectors (AAV), herpes simplex, pox virus, hybrid adenoviral vector, epstein-bar virus, lentivirus, or herpes simplex virus.

In certain embodiments, the AAV vectors as described herein can be derived from any AAV. In certain embodiments, the AAV vector is derived from the defective and nonpathogenic parvovirus adeno-associated type 2 virus. All such vectors are derived from a plasmid that retains only the AAV 145 bp inverted terminal repeats flanking the transgene expression cassette. Efficient gene transfer and stable transgene delivery due to integration into the genomes of the transduced cell are key features for this vector system. (Wagner et al., Lancet 351:9117 1702-3, 1998; Kearns et al., Gene Ther. 9:748-55, 1996). Other AAV serotypes, including AAV1, AAV2, AAV3, AAV4, AAV5, AAV6, AAV7, AAV8, AAV9 and AAVrh.10 and any novel AAV serotype can also be used in accordance with the present invention. In some embodiments, chimeric AAV is used where the viral origins of the long terminal repeat (LTR) sequences of the viral nucleic acid are heterologous to the viral origin of the capsid sequences. Non-limiting examples include chimeric virus with LTRs derived from AAV2 and capsids derived from AAV5, AAV6, AAV8 or AAV9 (i.e. AAV2/5, AAV2/6, AAV2/8 and AAV2/9, respectively).

The constructs described herein may also be incorporated into an adenoviral vector system. Adenoviral based vectors are capable of very high transduction efficiency in many cell types and do not require cell division. With such vectors, high titer and high levels of expression can been obtained.

The methods and compositions described herein can be used in a variety of cells, including liver cells, endothelial cells, lung cells, blood cells, and pancreas cells. The methods and compositions of the invention can also be used in the production of modified organisms. The modified organisms can be small mammals, companion animals, livestock, and primates. Non-limiting examples of rodents may include mice, rats, hamsters, gerbils, and guinea pigs. Non-limiting examples of companion animals may include cats, dogs, rabbits, hedgehogs, and ferrets. Non-limiting examples of livestock may include horses, goats, sheep, swine, llamas, alpacas, and cattle. Non-limiting examples of primates may include capuchin monkeys, chimpanzees, lemurs, macaques, marmosets, tamarins, spider monkeys, squirrel monkeys, and vervet monkeys. In one embodiment, the methods and compositions described herein can be used in mouse models with non-functional vWF genes (Denise et al., PNAS 95:9524-9529, 1998).

The methods and compositions described herein can be used to facilitate transgene integration in an endogenous vWF gene. Integration can occur through homologous recombination or non-homologous end joining. To facilitate homologous recombination between the vWF gene and a donor molecule, the donor molecule can contain sequence that is homologous to the vWF gene (e.g., exhibiting between about 80 to 100% sequence identity). To further facilitate homologous recombination, a double-strand break or single-strand nick can be introduced into the endogenous vWF gene. The double-strand break or single-strand nick can be introduced using one or more rare-cutting endonucleases either in nuclease or nickase formats. The double-strand break or single-strand nicks can be introduced at the site where integration is desired, or a distance upstream or downstream of the site. The distance from the integration site and the double-strand break (or single-strand nick) can be between 0 bp and 10,000 bp.

The methods and compositions described herein can be used to facilitate homology-independent insertion of a transgene into an endogenous vWF gene. In one embodiment, a transgene can harbor a partial coding sequence of the vWF gene and flanking rare-cutting endonuclease target sites can be administered to a cell. Following cleavage by the rare-cutting endonuclease, the liberated transgene can be captured during the repair of a double-strand break and integrated within an endogenous vWF gene. In another embodiment, a linear transgene harboring a partial coding sequence of the vWF gene can be administered to a cell. The linear transgene can be captured during the repair of a double-strand break and integrated within an endogenous vWF gene.

The methods described in this document can include the use of rare-cutting endonucleases for stimulating recombination or integrating the donor molecule into the vWF gene. The rare-cutting endonuclease can include CRISPR, TALENs, or zinc-finger nucleases (ZFNs). The CRISPR system can include CRISPR/Cas9 or CRISPR/Cpf1/Cas12a. The CRISPR system can include variants which display broad PAM capability (Hu et al., Nature 556, 57-63, 2018; Nishimasu et al., Science DOI: 10.1126, 2018) or higher on-target binding or cleavage activity (Kleinstiver et al., Nature 529:490-495, 2016). The rare-cutting endonuclease can be in the format of a nuclease (Mali et al., Science 339:823-826, 2013; Christian et al., Genetics 186:757-761, 2010), nickase (Cong et al., Science 339:819-823, 2013; Wu et al., Biochemical and Biophysical Research Communications 1:261-266, 2014), CRISPR-FokI dimers (Tsai et al., Nature Biotechnology 32:569-576, 2014), or paired CRISPR nickases (Ran et al., Cell 154:1380-1389, 2013).

The methods described in this document can also include the use of transposases for stimulating integration of the partial coding sequence into the vWF gene. The transposase can include a CRISPR-associated transposase (Strecker et al., Science 10.1126/science.aax9181, 2019; Klompe et al., Nature, 10.1038/s41586-019-1323-z, 2019). The transposases can be used in combination with a transgene comprising a transposon left end and right end. The CRISPR transposases can include the TypeV-U5, C2C5 CRISPR protein, Cas12k, along with proteins tnsB, tnsC, and tniQ. In some embodiments, the Cas12k can be from Scytonema hofmanni (SEQ ID NO:21) or Anabaena cylindrica (SEQ ID NO:22). Alternatively, the CRISPR transposase can include the Cas6 protein, along with helper proteins including Cas7, Cas8 and TniQ.

The methods and compositions provided herein can be used within to modify endogenous genes within cells. The endogenous genes can include, fibrinogen, prothrombin, tissue factor, Factor V, Factor VII, Factor VIII, Factor IX, Factor X, Factor XI, Factor XII (Hageman factor), Factor XIII (fibrin-stabilizing factor), von Willebrand factor, prekallikrein, high molecular weight kininogen (Fitzgerald factor), fibronectin, antithrombin III, heparin cofactor II, protein C, protein S, protein Z, protein Z-related protease inhibitor, plasminogen, alpha 2-antiplasmin, tissue plasminogen activator, urokinase, plasminogen activator inhibitor-1, plasminogen activator inhibitor-2, glucocerebrosidase (GBA), α-galactosidase A (GLA), iduronate sulfatase (IDS), iduronidase (IDUA), acid sphingomyelinase (SMPD1), MMAA, MMAB, MMACHC, MMADHC (C2orf25), MTRR, LMBRD1, MTR, propionyl-CoA carboxylase (PCC) (PCCA and/or PCCB subunits), a glucose-6-phosphate transporter (G6PT) protein or glucose-6-phosphatase (G6Pase), an LDL receptor (LDLR), ApoB, LDLRAP-1, a PCSK9, a mitochondrial protein such as NAGS (N-acetylglutamate synthetase), CPS1 (carbamoyl phosphate synthetase I), and OTC (ornithine transcarbamylase), ASS (argininosuccinic acid synthetase), ASL (argininosuccinase acid lyase) and/or ARGI (arginase), and/or a solute carrier family 25 (SLC25A13, an aspartate/glutamate carrier) protein, a UGT1A1 or UDP glucuronsyltransferase polypeptide A1, a fumarylacetoacetate hydrolyase (FAH), an alanine-glyoxylate aminotransferase (AGXT) protein, a glyoxylate reductase/hydroxypyruvate reductase (GRHPR) protein, a transthyretin gene (TTR) protein, an ATP7B protein, a phenylalanine hydroxylase (PAH) protein, and a lipoprotein lyase (LPL) protein.

The methods described herein can include the modification of the N- and C-terminus of genes associated with genetic disorders Gaucher disease, Hunter Syndrome, Fabry disease, Pompe disease, Maroteaux-Lamy syndrome, Morquio A syndrome, Lysosomal acid lipase deficiency, Hemophilia A, Hemophilia B, Hemophilia C, and Von Willebrand disease. The N-terminal modification can include replacement of at least the first coding exon but up to the penalutimate exon, along with insertion of a promoter and splice donor. The sequence can be inserted into the endogenous exon that encodes a homologous peptide sequence to the last exon in the partial coding sequence. Also, the sequence can be inserted into the intron following the endogenous exon that encodes a homologous peptide sequence to the last exon in the partial coding sequence. The C-terminal modification can include replacement of at least the last exon, but up to the second coding exon, along with insertion of a terminator and splice acceptor. The sequence can be inserted into the endogenous intron directly before the endogenous exon that encodes a homologous peptide sequence to the first exon in the partial coding sequence.

In one embodiment, the modification for Gaucher disease can include the insertion of a promoter and partial coding sequence and splice donor into GBA gene. The GBA gene comprises 12 exons. The partial coding sequence can contain exon 1, exons, exons 1-3, exons 1-4, exons 1-5, exons 1-6, exons 1-7, exons 1-8, exons 1-9, exons 1-10, or exons 1-11, or the partial coding sequence can contain sequence that encodes the peptide produced by the endogenous GBA gene's exon 1, exons, exons 1-3, exons 1-4, exons 1-5, exons 1-6, exons 1-7, exons 1-8, exons 1-9, exons 1-10, or exons 1-11. The modification can occur in hepatocytes. In another embodiment, the modification for Gaucher disease can include the insertion of a terminator, splice acceptor and partial coding sequence into the GBA gene. The partial coding sequence can contain exon 12, exons 11-12, exons 10-12, exons 9-12, exons 8-12, exons 7-12, exons 6-12, exons 5-12, exons 4-12, exons 3-12, or exons 2-12.

In another embodiments, the modification can target the IDS gene (Hunter Syndrome), GLA gene (Fabry disease), GAA gene (Pompe disease), ARSB gene (Maroteaux-Lamy syndrome), GALNS gene (Morquio A syndrome), GLB1 gene (Morquio A syndrome), LIPA gene (Lysosomal acid lipase deficiency), F8 gene (Hemophilia A), F9 gene (Hemophilia B), F11 gene (Hemophilia C), and vWF gene (Von Willebrand disease). The modification can include the N′ terminus of the endogenous protein through integrating a promoter, partial coding sequence and splice donor into the endogenous gene. The modification can occur in hepatocytes.

The transgene may include sequence for modifying the sequence encoding a polypeptide that is lacking or non-functional or having a gain-of-function mutation in the subject having a genetic disease, including but not limited to the following genetic diseases: achondroplasia, achromatopsia, acid maltase deficiency, adenosine deaminase deficiency, adrenoleukodystrophy, aicardi syndrome, alpha-1 antitrypsin deficiency, alpha-thalassemia, androgen insensitivity syndrome, pert syndrome, arrhythmogenic right ventricular dysplasia, ataxia telangictasia, barth syndrome, beta-thalassemia, blue rubber bleb nevus syndrome, canavan disease, chronic granulomatous diseases (CGD), cri du chat syndrome, cystic fibrosis, dercum's disease, ectodermal dysplasia, fanconi anemia, fibrodysplasia ossificans progressive, fragile X syndrome, galactosemis, Gaucher's disease, generalized gangliosidoses (e.g., GM1), hemochromatosis, the hemoglobin C mutation in the 6th codon of beta-globin (HbC), hemophilia, Huntington's disease, Hurler Syndrome, hypophosphatasia, Klinefleter syndrome, Krabbes Disease, Langer-Giedion Syndrome, leukocyte adhesion deficiency, leukodystrophy, long QT syndrome, Marfan syndrome, Moebius syndrome, mucopolysaccharidosis (MPS), nail patella syndrome, nephrogenic diabetes insipdius, neurofibromatosis, Neimann-Pick disease, osteogenesis imperfecta, porphyria, Prader-Willi syndrome, progeria, Proteus syndrome, retinoblastoma, Rett syndrome, Rubinstein-Taybi syndrome, Sanfilippo syndrome, severe combined immunodeficiency (SCID), Shwachman syndrome, sickle cell disease (sickle cell anemia), Smith-Magenis syndrome, Stickler syndrome, Tay-Sachs disease, Thrombocytopenia Absent Radius (TAR) syndrome, Treacher Collins syndrome, trisomy, tuberous sclerosis, Turner's syndrome, urea cycle disorder, von Hippel-Landau disease, Waardenburg syndrome, Williams syndrome, Wilson's disease, Wiskott-Aldrich syndrome, X-linked lymphoproliferative syndrome, lysosomal storage diseases (e.g., Gaucher's disease, GM1, Fabry disease and Tay-Sachs disease), mucopolysaccahidosis (e.g. Hunter's disease, Hurler's disease), hemoglobinopathies (e.g., sickle cell diseases, HbC, α-thalassemia, β-thalassemia) and hemophilias. Additional diseases that can be treated by targeted integration include von Willbrand disease, usher syndrome, polycystic kidney disease, spinocerebellar ataxia type 3, and spinocerebellar ataxia type 6.

The methods and compositions described in this document can be used in any circumstance where it is desired to modify the coding sequence of an endogenous gene. This technology is particularly useful for genes with coding sequences that exceed the size capacity of vectors or methods which delivery nucleic acids to cells. Furthermore, the methods and compositions described herein are useful in patients with mutations in the vWF gene. For example, patients with mutations in exons 18-20 (e.g., vWD type 2N) could benefit from the replacement of the 5′ end of the endogenous vWF coding sequence with a synthetic and WT vWF coding sequence. In another example, patients with mutations in exon 42 (e.g., vWD type 3) could benefit from the replacement of the 3′ end of the endogenous vWF coding sequence with a synthetic and WT vWF coding sequence.

The methods and compositions described in this document can also be used in the production of transgenic organisms or transgenic animals. Transgenic animals can include those developed for disease models, as well as animals with desirable traits. Cells within the animals can be used in combination with the methods and compositions described herein, which includes embryos. The animals can include small mammals (e.g., mice, rats, hamsters, gerbils, guinea pigs, rabbits, etc.), companion animals (e.g., dogs, cats, rabbits, hedgehogs and ferrets), livestock (horses, goats, sheep, swine, llamas, alpacas, and cattle), primates (capuchin monkeys, chimpanzees, lemurs, macaques, marmosets, tamarins, spider monkeys, squirrel monkeys, and vervet monkeys), and humans.

The invention will be further described in the following examples, which do not limit the scope of the invention described in the claims.

### EXAMPLES

### Example 1—Modification of the N-Terminus of the vWF Protein in Human Cells

The endogenous human vWF coding sequence (5′ end) was targeted for modification. Three donor molecules were generated to insert a strong constitutive promoter followed by a partial vWF coding sequence and splice donor sequence. The construct was designed with arms of homology to facilitate integration by homologous recombination. The first vector, pBA1100-D1, contained a CMV promoter followed by vWF exons 2-20 and a splice donor sequence. The sequences were flanked by a 646 bp left homology arm and an 861 bp right homology arm. The vector sequence is shown in SEQ ID NO:9 (Table 1) and the corresponding CRISPR nuclease target site is shown in SEQ ID NO:12 (Table 2). To prevent Cas9 from cutting the construct, a synonymous single nucleotide change was included in the PAM sequence. The second vector, pBA1102-D1, contained a CMV promoter followed by vWF exons 2-22 and a splice donor sequence. The sequences were flanked by a 372 bp left homology arm and an 853 bp right homology arm. The vector sequence is shown in SEQ ID NO:10 and the corresponding CRISPR nuclease target site is shown in SEQ ID NO:13. To prevent Cas9 from cutting the construct, a synonymous single nucleotide change was included in the PAM sequence. The third vector, pBA1104-D1, contained a CMV promoter followed by vWF exons 2-27 and a splice donor sequence. The sequences were flanked by a 350 bp left homology arm and a 400 bp right homology arm. The vector sequence is shown in SEQ ID NO:11 and the corresponding CRISPR nuclease target site is shown in SEQ ID NO:14. To prevent Cas9 from cutting the construct, a synonymous single nucleotide change was included in the PAM sequence.

TABLE 1
Donor molecules for integration within the 5′ end of
the human vWF gene

| | | vWF | | |
|---|---|---|---|---|
| Name | Promoter | exons | Site of integration | SEQ ID NO: |
| pBA1100-D1 | CMV | 2-20 | Following exon 20 | 9 |
| pBA1102-D1 | CMV | 2-22 | Following exon 22 | 10 |
| pBA1104-D1 | CMV | 2-27 | Following exon 27 | 11 |

TABLE 2
CRISPR/Cas9 target sites for targeting
double-strand DNA breaks within
the 5′ end of the human vWF gene

| Name | Target | PAM | SEQ ID NO: |
|---|---|---|---|
| pBA1101-C1 | CAGGTATTTGAGCCCGTCGA | AGG | 12 |
| pBA1103-C1 | GCTGGGCAAAGCCCTCTCCG | TGG | 13 |
| pBA1105-C1 | TCTTTCCTGAGGCAAAACGC | CGG | 14 |

CRISPR nucleases, both Cas9 and the gRNA, were generated as RNA and verified for activity in HEK293T cells. CRISPR RNA was delivered to cells by electroporation (Neon electroporation) and gene editing efficiencies were tested by sequence trace decomposition (Brinkman et al., Nucleic Acids Research 42:e168, 2014). Nuclease pBA1101-C1 had approximately 20% activity; nuclease pBA1103-C1 had approximately 10% activity; and nuclease pBA1105-C1 had approximately 20% activity.

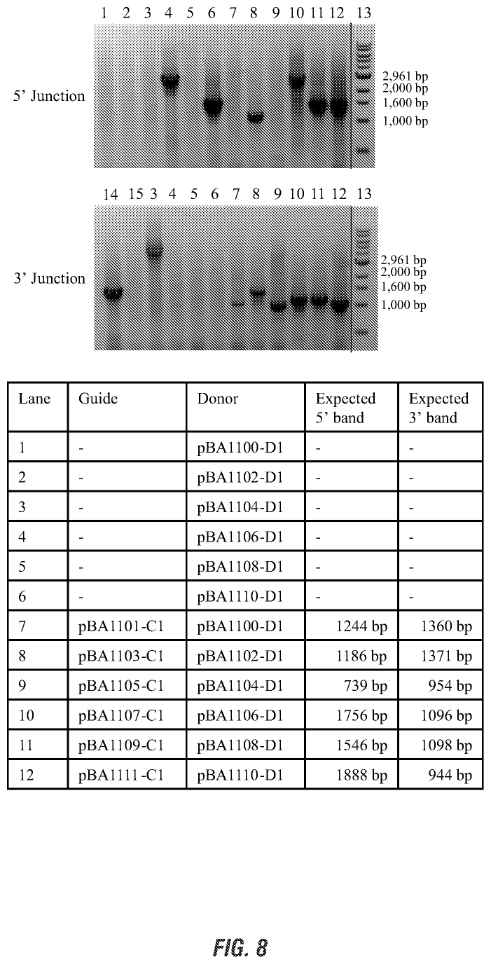

To knockin the vWF transgenes in the endogenous vWF gene, both the CRISPR RNA and donor molecules were transfected into HEK293T cells by electroporation. 72 hours post transfection, genomic DNA was isolated. Successful integration of the vWF transgene was verified by PCR (FIG. 8). Primers were designed to detect the 5′ and 3′ junctions. To detect the 5′ junction of the transgene carried on pBA1100-D1, primers (TGTATTTCTGTTCAGGGAGATGG; SEQ ID NO:25) and (AGATGTACTGCCAAGTAGGAAAG; SEQ ID NO:26) were used. To detect the 3′ junction of the transgene carried on pBA1100-D1, primers (CCATCACACCATGTGCTACT; SEQ ID NO:27) and (TCCATTCAGACCACACCAAG; SEQ ID NO:28) were used. To detect the 5′ junction of the transgene carried on pBA1102-D1, primers (GGGATGGGAGGTGAATTCTT; SEQ ID NO:30) and (AGATGTACTGCCAAGTAGGAAAG; SEQ ID NO:26) were used. To detect the 3′ junction of the transgene carried on pBA1102-D1, primers (ACGTTCTGGTGCAGGATTAC; SEQ ID NO:31) and (TGGCCCATGACTCAATGATAAG; SEQ ID NO:32) were used. To detect the 5′ junction of the transgene carried on pBA1104-D1, primers (CCGATAGAACTTTCTGCAGTGG; SEQ ID NO:33) and (AGATGTACTGCCAAGTAGGAAAG; SEQ ID NO:26) were used. To detect the 3′ junction of the transgene carried on pBA1104-D1, primers (CTGTAGAATCCTTACCAGTGACG; SEQ ID NO:34) and (CCTGCCACCTTGACTATGG; SEQ ID NO:35) were used. The data shows integration of the pBA1102 and pBA1104 transgenes within the endogenous vWF gene (FIG. 8).

To verify expression of the modified vWF gene, cDNA was prepared from the population of modified cells. Primers were designed to specifically detect expression from the modified vWF gene. Primers were designed to bind to the single-nucleotide polymorphisms present within the modified CRISPR target site. To avoid detecting genomic DNA, primers were designed to span an intron. Expression was normalized to an internal control (GAPDH). The results suggest that expression of the modified vWF gene occurred from targeted integration of pBA1102 and pBA1104.

### Example 2—Modification of the C-Terminus of the vWF Protein in Human Cells

The endogenous human vWF coding sequence (3′ end) was targeted for modification. Three donor molecules were generated to insert a partial vWF coding sequence followed by a transcriptional terminator. The construct was designed with arms of homology to facilitate integration by homologous recombination. The first vector, pBA1106-D1, contained a splice acceptor sequence, vWF exons 35-52, and a SV40 terminator. The sequences were flanked by a 1200 bp left homology arm and a 757 bp right homology arm. The vector sequence is shown in SEQ ID NO:15 (Table 5) and the corresponding CRISPR nuclease target site is shown in SEQ ID NO:18 (Table 6). To prevent Cas9 from cutting the construct, three synonymous single nucleotide change were included in the binding sequence. The second vector, pBA1108-D1, contained a splice acceptor sequence, vWF exons 33-52, and a SV40 terminator. The sequences were flanked by a 1001 bp left homology arm and a 734 bp right homology arm. The vector sequence is shown in SEQ ID NO:16 and the corresponding CRISPR nuclease target site is shown in SEQ ID NO:19. To prevent Cas9 from cutting the construct, a synonymous single nucleotide change was included in the PAM sequence. The third vector, pBA1110-D1, contained a splice acceptor sequence, vWF exons 29-52, and a SV40 terminator. The sequences were flanked by a 900 bp left homology arm and a 468 bp right homology arm. The vector sequence is shown in SEQ ID NO:17 and the corresponding CRISPR nuclease target site is shown in SEQ ID NO:20. To prevent Cas9 from cutting the construct, two synonymous single nucleotide changes were included in the Cas9 binding sequence.

TABLE 3
Donor molecules for integration within the 3′ end of
the human vWF gene

| | | vWF | | |
|---|---|---|---|---|
| Name | Promoter | exons | Site of integration | SEQ ID NO: |
| pBA1106-D1 | CMV | 35-52 | Before exon 35 | 15 |
| pBA1108-D1 | CMV | 33-52 | Before exon 33 | 16 |
| pBA1110-D1 | CMV | 29-52 | Before exon 29 | 17 |

TABLE 4
CRISPR/Cas9 target sites for targeting
double-strand DNA breaks within
the 3′ end of the human vWF gene

| Name | Target | PAM | SEQ ID NO: |
|---|---|---|---|
| pBA1107-C1 | AAAGGTCACGATGTGCCGAG | TGG | 18 |
| pBA1109-C1 | GGATTTGCATGGATGAGGAT | GGG | 19 |
| pBA1111-C1 | TGAAATGAAGAGTTTCGCCA | AGG | 20 |

CRISPR nucleases, both Cas9 and the gRNA, were generated as RNA and verified for activity in HEK293T cells. CRISPR RNA was delivered to cells by electroporation (Neon electroporation) and gene editing efficiencies were tested by sequence trace decomposition (Brinkman et al., Nucleic Acids Research 42:e168, 2014). Nuclease pBA1107-C1 had approximately 20% activity and nuclease pBA11011-C1 had approximately 40% activity.

To knockin the vWF transgenes in the endogenous vWF gene, both the CRISPR RNA and donor molecules were transfected into HEK293T cells by electroporation. 72 hours post transfection, genomic DNA was isolated. Successful integration of the vWF transgene was verified by PCR (FIG. 8). Primers were designed to detect the 5′ and 3′ junction. To detect the 5′ junction of the transgene carried on pBA1106-D1, primers (TATGCAGAGGAGATAGGAGAGG; SEQ ID NO:36) and (GATCCCACACAGACCATACG; SEQ ID NO:37) were used. To detect the 3′ junction of the transgene carried on pBA1106-D1, primers (GCATTCTAGTTGTGGTTTGTCC; SEQ ID NO:38) and (GTGTCTCCAAGAGCATCTAGC; SEQ ID NO:39) were used. To detect the 5′ junction of the transgene carried on pBA1108-D1, primers (GTGCCCATGCATAAGATTTGG; SEQ ID NO:40) and (CCAGTCAGCTTGAAATTCTGC; SEQ ID NO:41) were used. To detect the 3′ junction of the transgene carried on pBA1108-D1, primers (GCATTCTAGTTGTGGTTTGTCC; SEQ ID NO:38) and (TGTTCAGCATAAAGGTTACAATCC; SEQ ID NO:42) were used. To detect the 5′ junction of the transgene carried on pBA1110-D1, primers (GATGTCAGGTGTCAGGTAGC; SEQ ID NO:43) and (CCAGTCAGCTTGAAATTCTGC; SEQ ID NO:41) were used. To detect the 3′ junction of the transgene carried on pBA1110-D1, primers (GCATTCTAGTTGTGGTTTGTCC; SEQ ID NO:38) and (ATGATCACTCCTGGACACAAAG; SEQ ID NO:44) were used. The data shows integration of the pBA1106, pBA1108 and pBA1110 transgenes within the endogenous vWF gene (FIG. 8).

### Example 3—Modification of the N-Terminus of the Mouse and Human vWF Proteins in Hepatocytes

The endogenous mouse vWF coding sequence (5′ end) is targeted for modification, specifically exons 1-20, 1-21 and 1-22. Three donor molecules are synthesized along with three CRISPR/Cas9 nucleases. The donor molecules are designed to harbor an hCMV-intron promoter upstream of a synthetic coding sequence for the 5′ end of the vWF gene and 600 bp homology arms. A list of the donor molecules is shown in Table 1.

TABLE 5
Donor molecules comprising transgenes for integration within
the 5′ end of the mouse vWF gene

| | | vWF | | |
|---|---|---|---|---|
| Name | Promoter | exons | Site of integration | SEQ ID NO: |
| pBA1001-D1 | hCMV-intron | 2-20 | Following exon 20 | 1 |
| pBA1002-D1 | hCMV-intron | 2-21 | Following exon 21 | 2 |
| pBA1003-D1 | hCMV-intron | 2-22 | Following exon 22 | 3 |

Three CRISPR/Cas9 vectors are designed to introduce double-strand breaks near the predicted site of integration for vectors pBA1001, pBA1002 and pBA1003. The gRNA targets are shown in Table 2.

TABLE 6
CRISPR/Cas9 target sites for targeting
double-strand DNA breaks within
the 5′ end of the mouse vWF gene

| Name | Target | PAM | SEQ ID NO: |
|---|---|---|---|
| pBA1001-C1 | TGTTCTGGTGCAGGTGAGAC | TGG | 4 |
| pBA1002-C1 | GGGGAGCTTGAACTGTTTGA | CGG | 5 |
| pBA1003-C1 | AGCAAGAAGGCCTGCTAACC | TGG | 6 |

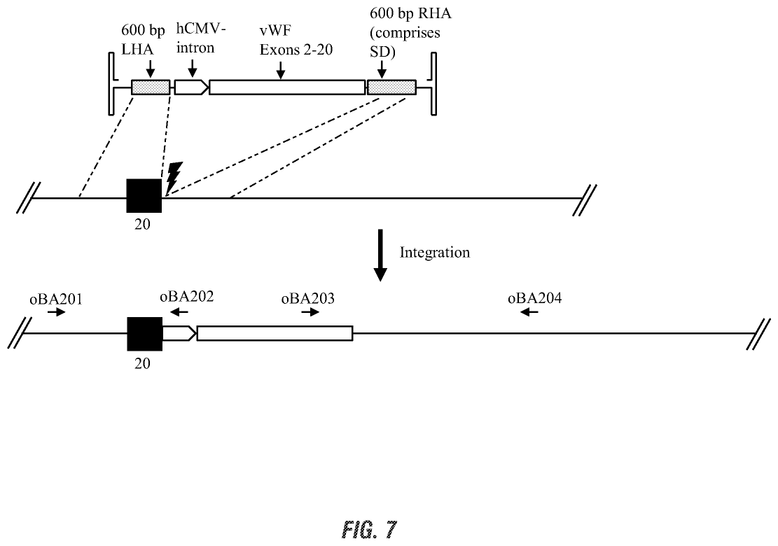

Confirmation of the function of the donor molecules and CRISPR/Cas9 vectors is achieved by transfection in murine hepatoma cells. Two days post transfection, DNA is extracted and assessed for mutations and targeted insertions within the vWF gene. Nuclease activity is analyzed using the Cel-I assay or by deep sequencing of amplicons comprising the CRISPR/Cas9 target sequence. Successful integration of the transgene is analyzed using the primers illustrated in FIG. 7.

To deliver the donor molecules (pBA1001-D1, pBA1002-D1, and pBA1003-D1) and CRISPR vectors (pBA1001-C1, pBA1002-C1, and pBA1003-C1) to liver cells in vivo the nucleic acid sequences are generated in hepatotropic adeno-associated virus vectors, serotype 8 (AAV8). Adult mice are treated by intravenous injection with 1×10¹¹ viral genomes per CRISPR viral vector and 5×10¹¹ viral genomes per donor viral vector per mouse (i.e., nuclease and donor molecules are mixed at a 1:5 ratio). Approximately two weeks after administration of the AAV vectors, mice are sacrificed and livers are harvested. The liver is used for DNA extraction, mRNA extraction and protein extraction using methods known in the art. Nuclease activity is analyzed using the Cel-I assay or by deep sequencing of amplicons comprising the CRISPR/Cas9 target sequence. Successful integration of the transgene is analyzed by PCR using the primers illustrated in FIG. 7.

A corresponding set of plasmids (both donor and CRISPR vectors) are generated targeting the insertion of exons 2-20, 2-21 and 2-22 into the human vWF gene. Human primary hepatocytes are transfected with AAV6 vectors harboring donor and CRISPR sequences. Two days post transfection, DNA is extracted. Nuclease activity is analyzed using the Cel-I assay or by deep sequencing of amplicons comprising the CRISPR/Cas9 target sequence. Successful integration of the transgene is analyzed by PCR.

### Example 4—Modification of the C-Terminus of the Mouse vWF Protein in Endothelial Cells

The mouse vWF coding sequence (3′ end) is targeted for modification, specifically exons 29-52. The cellular target for modification is endothelial cells. A donor molecule (pBA1004-D1; SEQ ID NO:7) is synthesized along with a corresponding CRISPR/Cas9 nuclease (pBA1004-C1). The donor molecule is designed to harbor a SV40 termination sequence downstream of a synthetic coding sequence comprising exons 29-52 of the vWF gene, wherein the SV40 termination sequence and coding sequence is flanked by 600 bp homology arms.

The CRISPR/Cas9 vector is designed to introduce a double-strand break near the predicted site of integration for vector pBA1004-D1. The target sequence for the gRNA, including the PAM sequence, is TGCAGACTGCAGCCAACCCCTGG (SEQ ID NO:8)

Confirmation of the function of the donor molecule pBA1004-D1 and CRISPR/Cas9 vectors is achieved by transfection in murine endothelial cells. Two days post transfection, DNA is extracted and assessed for mutations and targeted insertions within the vWF gene. Nuclease activity is analyzed using the Cel-I assay or by deep sequencing of amplicons comprising the CRISPR/Cas9 target sequence. Successful integration of the transgene is analyzed using primers within the transgene and within the endogenous vWF gene (but outside of the extent of the homology arms).

To deliver the donor molecule and CRISPR vector to endothelial cells in vivo, the nucleic acid sequences are generated in hepatotropic adeno-associated virus vectors, serotype 1 (AAV1). Adult mice are treated by intravenous injection with 1×10¹¹ viral genomes per CRISPR viral vector and 5×10¹¹ viral genomes per donor viral vector per mouse (i.e., nuclease and donor molecules are mixed at a 1:5 ratio). Approximately two weeks after administration of the AAV vectors, mice are sacrificed and vascular endothelial cells are harvested (Choi et al., Korean J Physiol Pharmacol. 19:35-42, 2015). The cells are used for DNA extraction, mRNA extraction and protein extraction using methods known in the art. Nuclease activity is analyzed using the Cel-I assay or by deep sequencing of amplicons comprising the CRISPR/Cas9 target sequence. Successful integration of the transgene is analyzed by PCR.

### Example 5—Modification of the N-Terminus of the vWF Protein in Human Cells Using CRISPR-Associated Transposases

CRISPR-associated transposase vectors, specifically ShCas12k, are designed to knockin the partial vWF transgenes carried on pBA1100, pBA1102 and pBA1104. To design the transgenes for use with ShCas12k, the homology arms are replaced with the left end (SEQ ID NO:23) and right end sequences (SEQ ID NO:24) of Cas12k transposons. Two vectors were generated: a vector comprising CMV promoters driving expression of tnsB, tnsC and tniQ, and a vector encoding ShCas12k (SEQ ID NO:21). Cas12k guide RNAs were designed to target sequences (GGGCTGGGAAGTCAGTCCCGCTC; SEQ ID NO:45), (GAATTGATCCCTTTACCATTATG; SEQ ID NO:46) and (TGAAGTGATGAATCTTATTGCTT; SEQ ID NO:47) for integration of pBA1100, pBA1102 and pBA1104 respectively.

To knockin the vWF transgenes in the endogenous vWF gene, the three vectors (ShCas12k, transposon, and tnsB/C/Q vectors) are transfected at equal molar concentrations into HEK293T cells by electroporation. 72 hours post transfection, genomic DNA is isolated and assessed for successful knockin by PCR.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of integrating a transgene into the von Willebrand factor gene, the method comprising:
a. administering a rare-cutting endonuclease or transposase targeted to a site within the von Willebrand factor gene, and
b. administering a transgene, wherein the transgene is integrated within the von Willebrand factor gene.

2. The method of claim 1, wherein the transposase comprises the Cas12k or Cas6 protein.

3. The method of claim 2, wherein the transposase comprises Cas12k from Scytonema hofmanni or Anabaena cylindrica.

4. The method of claim 1, wherein the rare-cutting endonuclease is selected from a CRISPR nuclease, TAL effector nuclease, zinc-finger nuclease, or meganuclease.

5. The method of claim 1, wherein the von Willebrand factor gene comprises a mutation that causes von Willebrand disease.

6. The method of any of claims 1-5, wherein the transgene comprises a promoter, a partial vWF coding sequence from a functional vWF gene, and a splice donor.

7. The method of claim 6, wherein the partial coding sequence comprises vWF exons 2-20, or encodes for the peptide produced by exons 2-20 of a functional vWF gene.

8. The method of claim 7, wherein the transgene is integrated in exon 20 or intron 20 of the aberrant vWF gene.

9. The method of claim 6, wherein the partial coding sequence comprises vWF exons 2-22, or encodes for the peptide produced by exons 2-22 of a functional vWF gene.

10. The method of claim 9, wherein the transgene is integrated in exon 22 or intron 22 of the vWF gene.

11. The method of claim 6, wherein the partial coding sequence comprises vWF exons 2-27, or encodes for the peptide produced by exons 2-27 of a functional vWF gene.

12. The method of claim 11, wherein the transgene is integrated in exon 27 or intron 27 of the vWF gene.

13. The method of claims 1-5, wherein the transgene comprises a splice acceptor, a partial vWF coding sequence from a functional vWF gene, and a terminator.

14. The method of claim 13, wherein the partial coding sequence comprises vWF exons 35-52, or encodes for the peptide produced by exons 35-52 of a functional vWF gene.

15. The method of claim 14, wherein the transgene is integrated in intron 34 of the vWF gene.

16. The method of claim 13, wherein the partial coding sequence comprises vWF exons 33-52, or encodes for the peptide produced by exons 33-52 of a functional vWF gene.

17. The method of claim 16, wherein the transgene is integrated in intron 32 of the vWF gene.

18. The method of claim 13, wherein the partial coding sequence comprises vWF exons 29-52, or encodes for the peptide produced by exons 29-52 of a functional vWF gene.

19. The method of claim 18, wherein the transgene is integrated in intron 28 of the vWF gene.

20. The method of any of claims 1-19, wherein the transgene comprises a left and right homology arm or a transposon left end and right end.

21. The method of any of claims 1-12 and 20, wherein the transgene is administered to a cell, and the cell is selected from a hepatocyte, an induced pluripotent stem cell (iPSC), a hematopoietic stem cell, a hepatic stem cell, or a red blood precursor cell.

22. The method of claim 21, wherein the cell is a hepatocyte.

23. The method of any of claims 1-5 and 13-19, wherein the transgene is administered to an endothelial cell.

24. The method of any of claims 22-23, wherein the transgene is harbored on an adeno-associated virus vector.

25. The method of claim 22, wherein the transgene is administered with lipid nanoparticles.

26. The method of claim 6, wherein the promoter is a tissue specific promoter, inducible promoter, or constitutive promoter.

27. The method of claim 26, wherein the promoter is an inducible promoter.


SEQUENCE LISTING (48 sequences)

SEQ ID NO: 1 - DNA, 4636 bp, Artificial Sequence
  Construct
ggcactctgtagcttttagggtgaggatatactctagcgagtccatggcacatggcttagggaggacgcctgccttctcttcacccacttcactccatatctctgtatcctcctgggaactcgagagcagcctggccttcctttctcctgcagactctgtgctttgaggcccatgtgccttccacaggctccaaatgcttggcttcttgaggctacctacgtgatgacctggcatcttgaactcaatctgtagaccaggctggccttgaactcagaaattcacctgcctctgcctgccaagtgctggtgtgtgtcaccacgcccggctttttatttattttattattattattattattattattattattattatttggtttgcctccttgtttcctaaggcctgattgaacccctggtgcctaaggtacaaaccagtaacacttgattgctgtccctagtgtctgccgggagcggaagtggaactgcacgaaccatgtgtgtgacgccacttgctctgccattggtatggcccactacctcaccttcgatggactcaagtacctgttcccgggggagtgccagtatgttctggtgcagagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgctgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcagatctttgtcgatcctaccatccactcgacacacccgccagcggccgcgttggtatcaaggttacaagacaggtttaaggagaccaatagaaactgggcatgtggagacagagaagactcttgggtttctgataggcactgactctcttcctttgtcctgttcccatttcagatgaaccctttcaggtatgagatctgcctgcttgttctggccctcacctggccagggaccctctgcacagaaaagccccgtgacaggccgtcgacggcccgatgcagcctctttggggacgacttcatcaacacgtttgatgagaccatgtacagctttgcagggggctgcagttatctcctggctggggactgccagaaacgttccttctccattctcgggaacttccaagatggcaagagaatgagcctgtctgtgtatcttggggagttttttgacatccatttgtttgccaatggcaccgtaacgcagggtgaccaaagcatctccatgccctacgcctcccaaggactctacctagaacgcgaggctgggtactataagctctccagtgagacctttggctttgcggccagaatcgatggcaatggcaacttccaagtcctgatgtcagacagacacttcaacaagacctgtgggctgtgcggtgattttaacatcttcgcggaagatgattttaggacgcaggaggggaccttgacctcagacccctatgattttgccaactcctgggccctgagcagtgaggaacagcggtgtaaacgggcatctcctcccagcaggaactgcgagagctcttctggggacatgcatcaggccatgtgggagcaatgccagctactgaagacggcatcggtgtttgcccgctgccaccctctggtggatcccgagtcctttgtggctctgtgtgagaagattttgtgtacgtgtgctacggggccagagtgcgcatgtcctgtactccttgagtatgcccgaacctgcgcccaggaagggatggtgctgtacggctggactgaccacagtgcctgtcgtccagcttgcccagctggcatggaatataaggagtgtgtgtctccttgccccagaacctgccagagcctgtctatcaatgaagtgtgtcagcagcaatgtgtagacggctgtagctgccctgagggagagctcttggatgaagaccgatgtgtgcagagctccgactgtccttgcgtgcacgctgggaagcggtaccctcctggcacctccctctctcaggactgcaacacttgtatctgcagaaacagcctatggatctgcagcaatgaggaatgcccaggggagtgtcttgtcacaggccaatcgcacttcaagagcttcgacaacaggtacttcaccttcagtgggatctgccaatatctgctggcccgggactgcgaggatcacactttctccattgtcatagagaccatgcagtgtgccgatgaccctgatgctgtctgcacccgctcggtcagtgtgcggctctctgccctgcacaacagcctggtgaaactgaagcacgggggagcagtgggcatcgatggtcaggatgtccagctccccttcctgcaaggtgacctccgcatccagcacacagtgatggcttctgtacgcctcagctatgcggaggacctgcagatggactgggatggccgtgggcggctactggttaagctgtccccagtctattctgggaagacctgtggcttgtgtgggaattacaacggcaacaagggagacgacttcctcacgccggccggcttggtggagcccctggtggtagacttcggaaacgcctggaagcttcaaggggactgttcggacctgcgcaggcaacacagcgacccctgcagcctgaatccacgcttgaccaggtttgcagaggaggcttgtgcgctcctgacgtcctccaagttcgaggcctgccaccacgcagtcagccctctgccctatctgcagaactgccgttatgatgtttgctcctgctccgacagccgggattgcctgtgtaacgcagtagctaactatgctgccgagtgtgcccgaaaaggcgtgcacatcgggtggcgggagcctggcttctgtgctctgggctgtccacagggccaggtgtacctgcagtgtgggaattcctgcaacctgacctgccgctccctctccctcccggatgaagaatgcagtgaagtctgtcttgaaggctgctactgcccaccagggctctaccaggatgaaagaggggactgtgtgcccaaggcccagtgcccctgctactacgatggtgagctcttccagcctgcggacattttctcagaccaccataccatgtgttactgtgaagatggcttcatgcactgtaccacaagtggcaccctggggagcctgttgcctgacactgtcctcagcagtcccctgtctcaccgtagcaaaaggagcctttcctgccggccacccatggtcaagctggtgtgtcctgctgacaacccacgggctcaagggctggagtgtgctaagacgtgccagaactacgacctggagtgtatgagcctgggctgtgtgtctggctgcctctgtcccccaggcatggtccggcacgaaaacaagtgtgtggccttggagcggtgtccctgcttccatcagggtgcagagtacgccccgggagacacagtgaagattggctgcaacacctgtgtctgccgggagcggaagtggaactgcacgaaccatgtgtgtgacgccacttgctctgccattggtatggcccactacctcaccttcgatggactcaagtacctgttcccgggggagtgccagtatgttctggtgcaggtgagactggaaatgaagagagggggtgttgtctgtgtcgggcagagcagggggtgccgtgggagtctctgggtcaggttctatctatagaaagacctttagggtttggttttgtcagagtaatttttttttaattcccccaaaggatgcccaacgatgtaggaagtttaagaacagaatttcattttctgagctggactggtatggtgctcctcaggccattttggcacaggtatagattagagaaagatgactctccattttggtggacattaaaccagagcacatgggagctgatcctggatgtcaccctccctgatagcccattgttaggaagtattcctgggcagggggattgcctgcatgacccctggagtgtgctgcctgacctatggtcctattgaagttgtcgccatttttatccttaacatggtgaggctggggctgcaggtgctgctcagtccagcaggaagaacatagagcatactatggagccccgccagcccctgtgcctcctcagacccaccttgtgggctcctgtgatttcttctgttggccgacaccacatagttcaccaaggaggacaagtgcttttctcag

SEQ ID NO: 2 - DNA, 4771 bp, Artificial Sequence
  Construct
atgtacaggtatgttcctgtgtgtgcatgtttatccgtgagtgtgtgtgtatgtttaggcatatatattatatgcttgtacatgtgtatgtgtgtatacatatgtatgtgtgtatgtatgtatatgcatgtggaagtatatatgaatgtgtgtttgtgtttgtatatgcatgtgtgcctatctgtgtatgtgtatgtttatatatgtatttgtatgtggatacatgtgctgtctcctctagcacccctgttttcctttgtgtatgcctctgtgctgagcgtgggtaccaaggtcatctgtaagggctgaggttagacccttgagagagtttccctggggctacctctccctttcccccatgtacagggtgcatctgatgatattctctgaccctggaagtcacgatgccatcttctgctctgtgagaaggggtaatcgttccctcttggtgccctctctccctaggattactgtggcagtaaccctgggacctttcagatcctggtgggaaatgagggttgcagctatccctcggtgaagtgcaggaagcgggtgaccatcctggtggatggaggggagcttgaactgtttgacggagagagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgctgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcagatctttgtcgatcctaccatccactcgacacacccgccagcggccgcgttggtatcaaggttacaagacaggtttaaggagaccaatagaaactgggcatgtggagacagagaagactcttgggtttctgataggcactgactctcttcctttgtcctgttcccatttcagatgaaccctttcaggtatgagatctgcctgcttgttctggccctcacctggccagggaccctctgcacagaaaagccccgtgacaggccgtcgacggcccgatgcagcctctttggggacgacttcatcaacacgtttgatgagaccatgtacagctttgcagggggctgcagttatctcctggctggggactgccagaaacgttccttctccattctcgggaacttccaagatggcaagagaatgagcctgtctgtgtatcttggggagttttttgacatccatttgtttgccaatggcaccgtaacgcagggtgaccaaagcatctccatgccctacgcctcccaaggactctacctagaacgcgaggctgggtactataagctctccagtgagacctttggctttgcggccagaatcgatggcaatggcaacttccaagtcctgatgtcagacagacacttcaacaagacctgtgggctgtgcggtgattttaacatcttcgcggaagatgattttaggacgcaggaggggaccttgacctcagacccctatgattttgccaactcctgggccctgagcagtgaggaacagcggtgtaaacgggcatctcctcccagcaggaactgcgagagctcttctggggacatgcatcaggccatgtgggagcaatgccagctactgaagacggcatcggtgtttgcccgctgccaccctctggtggatcccgagtcctttgtggctctgtgtgagaagattttgtgtacgtgtgctacggggccagagtgcgcatgtcctgtactccttgagtatgcccgaacctgcgcccaggaagggatggtgctgtacggctggactgaccacagtgcctgtcgtccagcttgcccagctggcatggaatataaggagtgtgtgtctccttgccccagaacctgccagagcctgtctatcaatgaagtgtgtcagcagcaatgtgtagacggctgtagctgccctgagggagagctcttggatgaagaccgatgtgtgcagagctccgactgtccttgcgtgcacgctgggaagcggtaccctcctggcacctccctctctcaggactgcaacacttgtatctgcagaaacagcctatggatctgcagcaatgaggaatgcccaggggagtgtcttgtcacaggccaatcgcacttcaagagcttcgacaacaggtacttcaccttcagtgggatctgccaatatctgctggcccgggactgcgaggatcacactttctccattgtcatagagaccatgcagtgtgccgatgaccctgatgctgtctgcacccgctcggtcagtgtgcggctctctgccctgcacaacagcctggtgaaactgaagcacgggggagcagtgggcatcgatggtcaggatgtccagctccccttcctgcaaggtgacctccgcatccagcacacagtgatggcttctgtacgcctcagctatgcggaggacctgcagatggactgggatggccgtgggcggctactggttaagctgtccccagtctattctgggaagacctgtggcttgtgtgggaattacaacggcaacaagggagacgacttcctcacgccggccggcttggtggagcccctggtggtagacttcggaaacgcctggaagcttcaaggggactgttcggacctgcgcaggcaacacagcgacccctgcagcctgaatccacgcttgaccaggtttgcagaggaggcttgtgcgctcctgacgtcctccaagttcgaggcctgccaccacgcagtcagccctctgccctatctgcagaactgccgttatgatgtttgctcctgctccgacagccgggattgcctgtgtaacgcagtagctaactatgctgccgagtgtgcccgaaaaggcgtgcacatcgggtggcgggagcctggcttctgtgctctgggctgtccacagggccaggtgtacctgcagtgtgggaattcctgcaacctgacctgccgctccctctccctcccggatgaagaatgcagtgaagtctgtcttgaaggctgctactgcccaccagggctctaccaggatgaaagaggggactgtgtgcccaaggcccagtgcccctgctactacgatggtgagctcttccagcctgcggacattttctcagaccaccataccatgtgttactgtgaagatggcttcatgcactgtaccacaagtggcaccctggggagcctgttgcctgacactgtcctcagcagtcccctgtctcaccgtagcaaaaggagcctttcctgccggccacccatggtcaagctggtgtgtcctgctgacaacccacgggctcaagggctggagtgtgctaagacgtgccagaactacgacctggagtgtatgagcctgggctgtgtgtctggctgcctctgtcccccaggcatggtccggcacgaaaacaagtgtgtggccttggagcggtgtccctgcttccatcagggtgcagagtacgccccgggagacacagtgaagattggctgcaacacctgtgtctgccgggagcggaagtggaactgcacgaaccatgtgtgtgacgccacttgctctgccattggtatggcccactacctcaccttcgatggactcaagtacctgttcccgggggagtgccagtatgttctggtgcaggattactgtggcagtaaccctgggacctttcagatcctggtgggaaatgagggttgcagctatccctcggtgaagtgcaggaagcgggtgaccatcctggtggatggaggggagcttgaactgtttgacggagaggtaagtgccagtctctcccctttacctttatgtccccttttgtccctccatttattaggagcggtttcccaatgttcatttagaactgagctggtagaatggcagccattgtagagaatgaggttgagctgtgttagctggtcttgagagtagaacaattgacagaccaatacaggccatgtgagcccattgcggttcaagtgtgggtgtgtgtgtgggagagggtctcctacagtgatctctgcctgtgtatcctcatacagtgatgactggtggaggtgagcagagtgtggcaggcaggaggggtttcccttccatgtacagtggtccccctcccttttagccacagagggataaaacccctgggagatgcctgagatcacaggcaatacagaacccagtgtgttccgttatgcatattggtaatgtcttagggcttctattacagtgacgaaacaccatgaccaaaaagcaagtgggggaggaagtggtttatttggcttacacttccacatcactgttcatcatcaaaggaagtcaggacaggaactcaaacaggacaggaacctggaggcaggagctgatgcagaagccatcaag

SEQ ID NO: 3 - DNA, 4918 bp, Artificial Sequence
  Construct
atggtagtcacttgggagaacagaggatggcaggaaaaaggaaaacaagtcgtgggggtgggtcataggtgggtagagtttttatcccattgactagaggtggttaaaatcatggccacattaaaaagcaagtcagggactggtgtagttggtgatagagggtcaaggcatcctccctcccttcttgtgaggtgacaggacgtgtgctggtctctttctctgtgttctcactgtggtcttgtcatcataggagggtgctaggtcagacagatgcagccattcctgtggagcggagggctggcctcagggagttctgctagaaagtagctggtggcacacacaggtctcaagggccatctctgctattggtgggcaaggcagacaggctgtgtgaaaagggagccctggactctgccctccttcacctcattttcccgtttcttcctccttcaggtgaacgttaagaggcccctgagagatgaatctcactttgaggtggtggagtcgggccggtacgtcatcctgctgctgggtcaggccctttctgtggtctgggaccaccacctcagcatctctgtggtcctgaagcacacataccagagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgctgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcagatctttgtcgatcctaccatccactcgacacacccgccagcggccgcgttggtatcaaggttacaagacaggtttaaggagaccaatagaaactgggcatgtggagacagagaagactcttgggtttctgataggcactgactctcttcctttgtcctgttcccatttcagatgaaccctttcaggtatgagatctgcctgcttgttctggccctcacctggccagggaccctctgcacagaaaagccccgtgacaggccgtcgacggcccgatgcagcctctttggggacgacttcatcaacacgtttgatgagaccatgtacagctttgcagggggctgcagttatctcctggctggggactgccagaaacgttccttctccattctcgggaacttccaagatggcaagagaatgagcctgtctgtgtatcttggggagttttttgacatccatttgtttgccaatggcaccgtaacgcagggtgaccaaagcatctccatgccctacgcctcccaaggactctacctagaacgcgaggctgggtactataagctctccagtgagacctttggctttgcggccagaatcgatggcaatggcaacttccaagtcctgatgtcagacagacacttcaacaagacctgtgggctgtgcggtgattttaacatcttcgcggaagatgattttaggacgcaggaggggaccttgacctcagacccctatgattttgccaactcctgggccctgagcagtgaggaacagcggtgtaaacgggcatctcctcccagcaggaactgcgagagctcttctggggacatgcatcaggccatgtgggagcaatgccagctactgaagacggcatcggtgtttgcccgctgccaccctctggtggatcccgagtcctttgtggctctgtgtgagaagattttgtgtacgtgtgctacggggccagagtgcgcatgtcctgtactccttgagtatgcccgaacctgcgcccaggaagggatggtgctgtacggctggactgaccacagtgcctgtcgtccagcttgcccagctggcatggaatataaggagtgtgtgtctccttgccccagaacctgccagagcctgtctatcaatgaagtgtgtcagcagcaatgtgtagacggctgtagctgccctgagggagagctcttggatgaagaccgatgtgtgcagagctccgactgtccttgcgtgcacgctgggaagcggtaccctcctggcacctccctctctcaggactgcaacacttgtatctgcagaaacagcctatggatctgcagcaatgaggaatgcccaggggagtgtcttgtcacaggccaatcgcacttcaagagcttcgacaacaggtacttcaccttcagtgggatctgccaatatctgctggcccgggactgcgaggatcacactttctccattgtcatagagaccatgcagtgtgccgatgaccctgatgctgtctgcacccgctcggtcagtgtgcggctctctgccctgcacaacagcctggtgaaactgaagcacgggggagcagtgggcatcgatggtcaggatgtccagctccccttcctgcaaggtgacctccgcatccagcacacagtgatggcttctgtacgcctcagctatgcggaggacctgcagatggactgggatggccgtgggcggctactggttaagctgtccccagtctattctgggaagacctgtggcttgtgtgggaattacaacggcaacaagggagacgacttcctcacgccggccggcttggtggagcccctggtggtagacttcggaaacgcctggaagcttcaaggggactgttcggacctgcgcaggcaacacagcgacccctgcagcctgaatccacgcttgaccaggtttgcagaggaggcttgtgcgctcctgacgtcctccaagttcgaggcctgccaccacgcagtcagccctctgccctatctgcagaactgccgttatgatgtttgctcctgctccgacagccgggattgcctgtgtaacgcagtagctaactatgctgccgagtgtgcccgaaaaggcgtgcacatcgggtggcgggagcctggcttctgtgctctgggctgtccacagggccaggtgtacctgcagtgtgggaattcctgcaacctgacctgccgctccctctccctcccggatgaagaatgcagtgaagtctgtcttgaaggctgctactgcccaccagggctctaccaggatgaaagaggggactgtgtgcccaaggcccagtgcccctgctactacgatggtgagctcttccagcctgcggacattttctcagaccaccataccatgtgttactgtgaagatggcttcatgcactgtaccacaagtggcaccctggggagcctgttgcctgacactgtcctcagcagtcccctgtctcaccgtagcaaaaggagcctttcctgccggccacccatggtcaagctggtgtgtcctgctgacaacccacgggctcaagggctggagtgtgctaagacgtgccagaactacgacctggagtgtatgagcctgggctgtgtgtctggctgcctctgtcccccaggcatggtccggcacgaaaacaagtgtgtggccttggagcggtgtccctgcttccatcagggtgcagagtacgccccgggagacacagtgaagattggctgcaacacctgtgtctgccgggagcggaagtggaactgcacgaaccatgtgtgtgacgccacttgctctgccattggtatggcccactacctcaccttcgatggactcaagtacctgttcccgggggagtgccagtatgttctggtgcaggattactgtggcagtaaccctgggacctttcagatcctggtgggaaatgagggttgcagctatccctcggtgaagtgcaggaagcgggtgaccatcctggtggatggaggggagcttgaactgtttgacggagaggtgaacgttaagaggcccctgagagatgaatctcactttgaggtggtggagtcgggccggtacgtcatcctgctgctgggtcaggccctttctgtggtctgggaccaccacctcagcatctctgtggtcctgaagcacacataccaggttagcaggccttcttgctgcttcttgcctgattcctgtggactgacatcagttctctaagaagtaacctgctgccctttcccagtcacattgggggacagtggttctctctctggtctagcctccttgctccccacacaagggaagctaagtagtcacagagggtgactgtacgtggggaggacagagacagctttgacagtgtcttgactagcccaggcaggcacacattttgttttcactgaggagggagagcaaggataggcagggtagcttttctttaggtttctaaacccacagaggcaaattaaatccacaaaatgttaaatcattgccatctattctgggatgttgttttaccagtgagcccaggctagcacacatgatcatgcacatgcttgtgtgtgtacatgaatgtgtgcatgtatgtgtgcatgtagaggccagccagtgggcctcatttctcaggagacatttactttgtgttttgagacaaagtctttcaccaggacctgggattgctcagcagttaggctgggctagctggctagtgagctttgaggacttgtttctgcctccccagcattggggtcacatatg

SEQ ID NO: 4 - DNA, 20 bp, Artificial Sequence
  CRISPR target site
tgttctggtgcaggtgagac

SEQ ID NO: 5 - DNA, 20 bp, Artificial Sequence
  CRISPR target site
ggggagcttgaactgtttga

SEQ ID NO: 6 - DNA, 20 bp, Artificial Sequence
  CRISPR target site
agcaagaaggcctgctaacc

SEQ ID NO: 7 - DNA, 4811 bp, Artificial Sequence
  Construct
tacttctctgtgaatgacacaacttccttctctcccaggcactgatgccacattttcttgactccttcaaaggtcacacacagcctgtttcccagttacatgctgtactgtctctttgcccttgtttcaaccagtcttagacccaagaacacagaagcagattttctttctcttattaattgtttagctaattctcagaattatgagtcttagaatgacattttcatatatatacatatacacatacacatacacatacatatacatatatatttttttcctggcccccttcttccccacaaacaacctcagttacttttcctgttatttagagagggcagcctcttgctctcatttgcagctatttgcactgtctgtgggtagagctccagtcttttcgatgactgtcaatctagtgagcccatagattcaggaactgtctcctctgtccttctacctgacccattcccatgccctgccctccctggcaaacacgtgctcagtggtgcactgaagaccactggctgttgtgggggctgacggctggccttccattagcacctgtgacttgtgtacccatgctcttgtttctctctgcagactgcagccaacccctggatgtggtcctgctcctggatggctcctctagcttgccagagtcttcctttgataaaatgaagagttttgccaaggctttcatttcaaaggccaacattgggccccacctcacacaggtgtccgtgatacagtatggaagcatcaataccattgatgtaccatggaatgtggttcaggagaaagcccatctacagagtttggtggacctcatgcagcaggagggtggccccagccagattggggatgctctggcctttgccgtgcgctatgtaacttcacaaatccacggagccaggcctggggcctccaaagcagtggtcatcatcatcatggatacctccttggatcccgtggacacagcagcagatgctgccagatccaaccgagtggcagtgtttcccgttggggttggggatcggtatgatgaagcccagctgaggatcttggcaggccctggggccagctccaatgtggtaaagctccagcaagttgaagacctctccaccatggccaccctgggcaactccttcttccacaaactgtgttctgggttttctggagtttgtgtggatgaagatgggaatgagaagaggcctggggatgtctggaccttgccggatcagtgccacacagtgacttgcttggcaaatggccagaccttgctgcagagtcatcgtgtcaattgtgaccatggaccccggccttcatgtgccaacagccagtctcctgttcgggtggaggagacgtgtggctgccgctggacctgcccttgtgtgtgcacgggcagttccactcggcacatcgtcaccttcgatgggcagaatttcaagcttactggtagctgctcctatgtcatctttcaaaacaaggagcaggacctggaagtgctcctccacaatggggcctgcagccccggggcaaaacaagcctgcatgaagtccattgagattaagcatgctggcgtctctgctgagctgcacagtaacatggagatggcagtggatgggagactggtccttgccccgtacgttggtgaaaacatggaagtcagcatctacggcgctatcatgtatgaagtcaggtttacccatcttggccacatcctcacatacacgccacaaaacaacgagttccaactgcagcttagccccaagacctttgcttcgaagatgcatggtctttgcggaatctgtgatgaaaacggggccaatgacttcacgttgcgagatggcacggtcaccacagactggaaaaggcttgtccaggaatggacggtgcagcagccagggtacacatgccaggctgttcccgaggagcagtgtcccgtctctgacagctcccactgccaggtcctcctctcagcgtcgtttgctgaatgccacaaggtcatcgctccagccacattccataccatctgccagcaagacagttgccaccaggagcgagtgtgtgaggtgattgcttcttacgcccatctctgtcggaccagtggggtctgtgttgattggaggacaactgatttctgtgctatgtcatgcccaccgtccctggtgtataaccactgtgagcgtggctgccctcggcactgcgatgggaacactagcttctgtggggaccatccctcagaaggctgcttctgtccccaacaccaagtttttctggaaggcagctgtgtccccgaggaggcctgcactcagtgtgttggcgaggatggagttcgacatcagttcctggagacctgggtcccagaccatcagccctgtcagatctgtatgtgcctcagtgggagaaagattaactgcactgcccagccgtgtcccacagcccgagctcccacgtgtggcccatgtgaagtggctcgcctcaagcagagcacaaacctgtgctgcccagagtatgagtgtgtgtgtgacctgttcaactgcaacttgcctccagtgcctccgtgtgaaggagggctccagccaaccctgaccaaccctggagaatgcagacccacctttacctgtgcctgcaggaaagaagagtgcaaaagagtgtccccaccctcctgcccccctcaccggacacccactctccggaagacccagtgctgtgatgaatacgagtgtgcttgcagctgtgtcaactccacgctgagctgcccacttggctacctggcctcagccactaccaatgactgtggctgcaccacgaccacctgtctccctgacaaggtttgtgtccaccgaggcaccgtctaccctgtgggccagttctgggaggagggctgtgacacgtgcacctgtacggacatggaggatactgtcgtgggcctgcgtgtggtccagtgctctcaaaggccctgtgaagacagctgtcagccaggtttttcttatgttctccacgaaggcgagtgctgtggaaggtgcctgccctctgcttgcaaggtggtggctggctcactgcggggcgattcccactcttcctggaaaagtgttggatctcggtgggctgttcctgagaacccctgcctcgtcaacgagtgtgtccgcgtggaggatgcagtgtttgtgcagcagaggaacatctcctgcccacagctggctgtccctacctgtcccacaggcttccaactgaactgtgagacctcagagtgctgtcctagctgccactgtgagcctgtggaggcctgcctgctcaatggcaccatcattgggcccgggaagagtgtgatggttgacctatgcacgacctgccgctgcatcgtgcagacagacgccatctccagattcaagctggagtgcaggaagactacctgtgaggcctgccccatgggctatcgggaagagaagagccagggtgaatgctgtgggagatgcttgcctacagcttgcactattcagctaagaggaggacggatcatgaccctgaagcaagatgagacattccaggatggctgtgacagtcatttgtgcagggtcaacgagagaggagagtacatctgggagaagagggtcacgggctgcccaccatttgatgaacacaagtgtctggctgaaggaggcaaaatcgtgaaaattccaggcacctgctgtgacacatgtgaggagcctgattgcaaagacatcacagccaaggtgcagtacatcaaagtgggagattgtaagtcccaagaggaagtggacattcattactgccagggaaagtgtgccagcaaagctgtgtactccattgacatcgaggatgtgcaggagcaatgctcctgctgcctgccctcgaggacggagcccatgcgcgtgcccttgcactgcaccaatggctctgtcgtgtaccacgaggtcatcaacgccatgcagtgcaggtgttctccccggaactgcagcaagtgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttactgcagccaacccctggatgtggtcctgctcctggatggctcctctagcttgccagagtcttcctttgataaaatgaagagttttgccaaggctttcatttcaaaggccaacattggtgagtgatacccttgaacctgcaggtgagggagtggctcttcctggttcattgattctaaatgtctcccttctccttttcctgttagggccccacctcacacaggtgtccgtgatacagtatggaagcatcaataccattgatgtaccatggaatgtggttcaggagaaagcccatctacagagtttggtggacctcatgcagcaggagggtggccccagccagattggtaatgcttggagccacgagctagatgtagaacttgtgttctgatcctcactcttgtgttctgattgagtgatcttgaccagtaactttactccttggtctgagtttctcttttgattggcgagaagctagatggtcctttgtgtcattttccagtcccaccaagtattgttctgagtcataactgctcatcttttgaatgtacctgagtcagcccttaagcccattgctcagagggtctagaatactccatcc

SEQ ID NO: 8 - DNA, 23 bp, Artificial Sequence
  CRISPR target site
tgcagactgcagccaacccctgg

SEQ ID NO: 9 - DNA, 4750 bp, Artificial Sequence
  Construct
gcttgaggtctgagcttaccacctctgacgcacaagtgggcttcttgatgtcagctctggttacgggtggtggcagggagggacacgtggcagtgggcagatcactatagattttaacatgtagctacatacatcttaacgtgtagctatgcacacaggactgctcctggcagaagtgcgtacttcatcactcttttctatactctgggctttcccactgttctgtcttgtttttcccattagcctcaggctttcaacatcagtgtgtctgttttacagacaccctgtggccaatctcaggtagatgtggctttcagggtgaggctgagcgaattcataacaggaggcctaaagagcatccgggcctcctccctggctgcctggctcactttggacaaccccttcccttctttgcctcagtttcccccttttagggacagccactaggcttccctgtctcctgctgggccccatgctgggcctatgaagtccacactccacgctacaggtcctcaacttccttgggcttcctggagggttgggaggcacccagagtattctgtgttccttcattgcctccatggcccagatgggcccctcaaacccaaggtgcccaacttgtcatctctgccatgactgctcctagcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaatgccaccatgattcctgccagatttgccggggtgctgcttgctctggccctcattttgccagggaccctttgtgcagaaggaactcgcggcaggtcatccacggcccgatgcagccttttcggaagtgacttcgtcaacacctttgatgggagcatgtacagctttgcgggatactgcagttacctcctggcagggggctgccagaaacgctccttctcgattattggggacttccagaatggcaagagagtgagcctctccgtgtatcttggggaattttttgacatccatttgtttgtcaatggtaccgtgacacagggggaccaaagagtctccatgccctatgcctccaaagggctgtatctagaaactgaggctgggtactacaagctgtccggtgaggcctatggctttgtggccaggatcgatggcagcggcaactttcaagtcctgctgtcagacagatacttcaacaagacctgcgggctgtgtggcaactttaacatctttgctgaagatgactttatgacccaagaagggaccttgacctcggacccttatgactttgccaactcatgggctctgagcagtggagaacagtggtgtgaacgggcatctcctcccagcagctcatgcaacatctcctctggggaaatgcagaagggcctgtgggagcagtgccagcttctgaagagcacctcggtgtttgcccgctgccaccctctggtggaccccgagccttttgtggccctgtgtgagaagactttgtgtgagtgtgctggggggctggagtgcgcctgccctgccctcctggagtacgcccggacctgtgcccaggagggaatggtgctgtacggctggaccgaccacagcgcgtgcagcccagtgtgccctgctggtatggagtataggcagtgtgtgtccccttgcgccaggacctgccagagcctgcacatcaatgaaatgtgtcaggagcgatgcgtggatggctgcagctgccctgagggacagctcctggatgaaggcctctgcgtggagagcaccgagtgtccctgcgtgcattccggaaagcgctaccctcccggcacctccctctctcgagactgcaacacctgcatttgccgaaacagccagtggatctgcagcaatgaagaatgtccaggggagtgccttgtcacaggtcaatcacacttcaagagctttgacaacagatacttcaccttcagtgggatctgccagtacctgctggcccgggattgccaggaccactccttctccattgtcattgagactgtccagtgtgctgatgaccgcgacgctgtgtgcacccgctccgtcaccgtccggctgcctggcctgcacaacagccttgtgaaactgaagcatggggcaggagttgccatggatggccaggacgtccagctccccctcctgaaaggtgacctccgcatccagcatacagtgacggcctccgtgcgcctcagctacggggaggacctgcagatggactgggatggccgcgggaggctgctggtgaagctgtcccccgtctatgccgggaagacctgcggcctgtgtgggaattacaatggcaaccagggcgacgacttccttaccccctctgggctggcggagccccgggtggaggacttcgggaacgcctggaagctgcacggggactgccaggacctgcagaagcagcacagcgatccctgcgccctcaacccgcgcatgaccaggttctccgaggaggcgtgcgcggtcctgacgtcccccacattcgaggcctgccatcgtgccgtcagcccgctgccctacctgcggaactgccgctacgacgtgtgctcctgctcggacggccgcgagtgcctgtgcggcgccctggccagctatgccgcggcctgcgcggggagaggcgtgcgcgtcgcgtggcgcgagccaggccgctgtgagctgaactgcccgaaaggccaggtgtacctgcagtgcgggaccccctgcaacctgacctgccgctctctctcttacccggatgaggaatgcaatgaggcctgcctggagggctgcttctgccccccagggctctacatggatgagaggggggactgcgtgcccaaggcccagtgcccctgttactatgacggtgagatcttccagccagaagacatcttctcagaccatcacaccatgtgctactgtgaggatggcttcatgcactgtaccatgagtggagtccccggaagcttgctgcctgacgctgtcctcagcagtcccctgtctcatcgcagcaaaaggagcctatcctgtcggccccccatggtcaagctggtgtgtcccgctgacaacctgcgggctgaagggctcgagtgtaccaaaacgtgccagaactatgacctggagtgcatgagcatgggctgtgtctctggctgcctctgccccccgggcatggtccggcatgagaacagatgtgtggccctggaaaggtgtccctgcttccatcagggcaaggagtatgcccctggagaaacagtgaagattggctgcaacacttgtgtctgtcgggaccggaagtggaactgcacagaccatgtgtgtgatgccacgtgctccacgatcggcatggcccactacctcacgttcgacgggctcaaatacctgttccccggggagtgccagtacgttctggtgcaggtgagaggtggggagatggggagagggtgctgtttctttctaggaggggtgggaggtgtggcctcaggttgggttctgtggatctgtctgcagaaacaactctggggtctggtttctactggagtacttcccagtccttcacagaagtgcctgaagcggtaggggatttgaagctcaaagtggttgtccattttccctctgctcacctggggacttataaaacgagacagaagcttgtttgttgttgaggattggtgtgggagaaaggctactgctagtccacattagcacagatgtggaattagaaaaagtcatctgttccttctggtagacacagcctcagtcagggtgcatagcttagggagtgggttgggctgggaagtcagtcccgctcagcctcccttccagcaccctgggcagtgcacagtctgcaggtgttgtgcagtggccctggacagggggatggttgaaatgacccctggagtttgcttcccacggtatggctttgtggaattctccgccattttaatgtctaacttggtacaattcagaatgggaggagtgggaggatgggacacaggaaagtcatcctgcccagcagatgagagcgatccaggaatcctcacggtgagtgtgggcagcagcccctctgcctcccactccccactgcgtggattcttgtaagtttctctttctggttgacatcaactgtgtaagcaaggaagtatgagtgcttttctcaccagagctgaggcactgtactctgtgaagctttgaacaaatatggtccctctgtctccattcccaggaggaggaggggcgggagcttggtgtggtctgaatggaagaccacaaacccat

SEQ ID NO: 10 - DNA, 4750 bp, Artificial Sequence
  Construct
agagatgcttaaaatcattgccatgttgaaaacctatctaggagaccactatatttaatgactaattgtcaataaaatacctgctcattggtttcatagtactttaatttcataatcatgattttgctgctacctctgttaccgtctcttggtcatggatgcctggagagtggtggtggtgagatggtcacagacatgtcctggcgtggggctggccctgcaggggtgcagtggcaggtggggtcctggaggggtggcagtgcctgcactcgtgggcactgaagacagatgggcaggtgtagagtggagggaggatctggctgtcgagcctgcccttcatcctcctggatttcttgctttgtcttcctccagcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaatgccaccatgattcctgccagatttgccggggtgctgcttgctctggccctcattttgccagggaccctttgtgcagaaggaactcgcggcaggtcatccacggcccgatgcagccttttcggaagtgacttcgtcaacacctttgatgggagcatgtacagctttgcgggatactgcagttacctcctggcagggggctgccagaaacgctccttctcgattattggggacttccagaatggcaagagagtgagcctctccgtgtatcttggggaattttttgacatccatttgtttgtcaatggtaccgtgacacagggggaccaaagagtctccatgccctatgcctccaaagggctgtatctagaaactgaggctgggtactacaagctgtccggtgaggcctatggctttgtggccaggatcgatggcagcggcaactttcaagtcctgctgtcagacagatacttcaacaagacctgcgggctgtgtggcaactttaacatctttgctgaagatgactttatgacccaagaagggaccttgacctcggacccttatgactttgccaactcatgggctctgagcagtggagaacagtggtgtgaacgggcatctcctcccagcagctcatgcaacatctcctctggggaaatgcagaagggcctgtgggagcagtgccagcttctgaagagcacctcggtgtttgcccgctgccaccctctggtggaccccgagccttttgtggccctgtgtgagaagactttgtgtgagtgtgctggggggctggagtgcgcctgccctgccctcctggagtacgcccggacctgtgcccaggagggaatggtgctgtacggctggaccgaccacagcgcgtgcagcccagtgtgccctgctggtatggagtataggcagtgtgtgtccccttgcgccaggacctgccagagcctgcacatcaatgaaatgtgtcaggagcgatgcgtggatggctgcagctgccctgagggacagctcctggatgaaggcctctgcgtggagagcaccgagtgtccctgcgtgcattccggaaagcgctaccctcccggcacctccctctctcgagactgcaacacctgcatttgccgaaacagccagtggatctgcagcaatgaagaatgtccaggggagtgccttgtcacaggtcaatcacacttcaagagctttgacaacagatacttcaccttcagtgggatctgccagtacctgctggcccgggattgccaggaccactccttctccattgtcattgagactgtccagtgtgctgatgaccgcgacgctgtgtgcacccgctccgtcaccgtccggctgcctggcctgcacaacagccttgtgaaactgaagcatggggcaggagttgccatggatggccaggacgtccagctccccctcctgaaaggtgacctccgcatccagcatacagtgacggcctccgtgcgcctcagctacggggaggacctgcagatggactgggatggccgcgggaggctgctggtgaagctgtcccccgtctatgccgggaagacctgcggcctgtgtgggaattacaatggcaaccagggcgacgacttccttaccccctctgggctggcggagccccgggtggaggacttcgggaacgcctggaagctgcacggggactgccaggacctgcagaagcagcacagcgatccctgcgccctcaacccgcgcatgaccaggttctccgaggaggcgtgcgcggtcctgacgtcccccacattcgaggcctgccatcgtgccgtcagcccgctgccctacctgcggaactgccgctacgacgtgtgctcctgctcggacggccgcgagtgcctgtgcggcgccctggccagctatgccgcggcctgcgcggggagaggcgtgcgcgtcgcgtggcgcgagccaggccgctgtgagctgaactgcccgaaaggccaggtgtacctgcagtgcgggaccccctgcaacctgacctgccgctctctctcttacccggatgaggaatgcaatgaggcctgcctggagggctgcttctgccccccagggctctacatggatgagaggggggactgcgtgcccaaggcccagtgcccctgttactatgacggtgagatcttccagccagaagacatcttctcagaccatcacaccatgtgctactgtgaggatggcttcatgcactgtaccatgagtggagtccccggaagcttgctgcctgacgctgtcctcagcagtcccctgtctcatcgcagcaaaaggagcctatcctgtcggccccccatggtcaagctggtgtgtcccgctgacaacctgcgggctgaagggctcgagtgtaccaaaacgtgccagaactatgacctggagtgcatgagcatgggctgtgtctctggctgcctctgccccccgggcatggtccggcatgagaacagatgtgtggccctggaaaggtgtccctgcttccatcagggcaaggagtatgcccctggagaaacagtgaagattggctgcaacacttgtgtctgtcgggaccggaagtggaactgcacagaccatgtgtgtgatgccacgtgctccacgatcggcatggcccactacctcaccttcgacgggctcaaatacctgttccccggggagtgccagtacgttctggtgcaggattactgcggcagtaaccctgggacctttcggatcctagtggggaataagggatgcagccacccctcagtgaaatgcaagaaacgggtcaccatcctggtggagggaggagagattgagctgtttgacggggaggtgaatgtgaagaggcccatgaaggatgagactcactttgaggtggtggagtctggccggtacatcattctgctgctgggcaaagccctctccgttgtctgggaccgccacctgagcatctccgtggtcctgaagcagacataccaggtcagtggctttcttgcttcatcttgttggggacttggcctttggagtgttttctgctccctgatcgtaggtctctaaggacttgctttatgaatccaggtgctcctgtgttgggtgcatatatatttaggatagttagctcttcttgttgaattgatccctttaccattatgtaatggccttcttttgatctttgttggttcaaagactgttttatcagatactaggattgcaacccctgcttttttttttttgccttccatttgcttggtagaccttcctccctccctttattttgagcctatgtatgtctctgcacgtgagatgggtctcctgaatacagcacactgatgggtcttgactctttatccaattggccagtctgtgccttttaattggggcatttagcccatttacatttaaggttaatattgtcatgtgtgaatttgatcctgtcattatgatgttcgctggttattttgcccattaattgataccgtttcttcgtagcatcgatggtctttacaatttggcatgtttttgcagtggctggtactggttgtttctttccatgtttagtgcttccttcaggagctcttgtaaggcaggtctggtggtgacaaaatctctcagcatttgcttgtctgtaaaggattttatttctctttcacttatgaagcttagtttgggtggatatgaaattctaagttgaaaattcttttctttaagaatgttgaatattggtccccccctctcttctggcttgtagggtttctgccaagagatctgctattagtctgatgggcttccctttgtgagtaactcgacatttctctctggctgcccttaacac

SEQ ID NO: 11 - DNA, 4982 bp, Artificial Sequence
  Construct
tagccacacgcagtttgtgacgatcctatggtacagcacagctctagagttactgaaggtgttattcagaagagcagaaagagccccggagataagatttcatttgtcctgaggcttggggaggtgaggtagggtgaaggaatccccgctcccagttttgcagagggatcaatcaaggcacaagcaggagagatgctccttgagtgatggggtgacccctgggagtgcaggcaggaggagttggcttctagggcaggaggaggagttggctcctcccttttagttaaaaatgaggcttcctcgtgggaaaggggagcgttttggttcctaatgagagctttcttttgcagcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaatgccaccatgattcctgccagatttgccggggtgctgcttgctctggccctcattttgccagggaccctttgtgcagaaggaactcgcggcaggtcatccacggcccgatgcagccttttcggaagtgacttcgtcaacacctttgatgggagcatgtacagctttgcgggatactgcagttacctcctggcagggggctgccagaaacgctccttctcgattattggggacttccagaatggcaagagagtgagcctctccgtgtatcttggggaattttttgacatccatttgtttgtcaatggtaccgtgacacagggggaccaaagagtctccatgccctatgcctccaaagggctgtatctagaaactgaggctgggtactacaagctgtccggtgaggcctatggctttgtggccaggatcgatggcagcggcaactttcaagtcctgctgtcagacagatacttcaacaagacctgcgggctgtgtggcaactttaacatctttgctgaagatgactttatgacccaagaagggaccttgacctcggacccttatgactttgccaactcatgggctctgagcagtggagaacagtggtgtgaacgggcatctcctcccagcagctcatgcaacatctcctctggggaaatgcagaagggcctgtgggagcagtgccagcttctgaagagcacctcggtgtttgcccgctgccaccctctggtggaccccgagccttttgtggccctgtgtgagaagactttgtgtgagtgtgctggggggctggagtgcgcctgccctgccctcctggagtacgcccggacctgtgcccaggagggaatggtgctgtacggctggaccgaccacagcgcgtgcagcccagtgtgccctgctggtatggagtataggcagtgtgtgtccccttgcgccaggacctgccagagcctgcacatcaatgaaatgtgtcaggagcgatgcgtggatggctgcagctgccctgagggacagctcctggatgaaggcctctgcgtggagagcaccgagtgtccctgcgtgcattccggaaagcgctaccctcccggcacctccctctctcgagactgcaacacctgcatttgccgaaacagccagtggatctgcagcaatgaagaatgtccaggggagtgccttgtcacaggtcaatcacacttcaagagctttgacaacagatacttcaccttcagtgggatctgccagtacctgctggcccgggattgccaggaccactccttctccattgtcattgagactgtccagtgtgctgatgaccgcgacgctgtgtgcacccgctccgtcaccgtccggctgcctggcctgcacaacagccttgtgaaactgaagcatggggcaggagttgccatggatggccaggacgtccagctccccctcctgaaaggtgacctccgcatccagcatacagtgacggcctccgtgcgcctcagctacggggaggacctgcagatggactgggatggccgcgggaggctgctggtgaagctgtcccccgtctatgccgggaagacctgcggcctgtgtgggaattacaatggcaaccagggcgacgacttccttaccccctctgggctggcggagccccgggtggaggacttcgggaacgcctggaagctgcacggggactgccaggacctgcagaagcagcacagcgatccctgcgccctcaacccgcgcatgaccaggttctccgaggaggcgtgcgcggtcctgacgtcccccacattcgaggcctgccatcgtgccgtcagcccgctgccctacctgcggaactgccgctacgacgtgtgctcctgctcggacggccgcgagtgcctgtgcggcgccctggccagctatgccgcggcctgcgcggggagaggcgtgcgcgtcgcgtggcgcgagccaggccgctgtgagctgaactgcccgaaaggccaggtgtacctgcagtgcgggaccccctgcaacctgacctgccgctctctctcttacccggatgaggaatgcaatgaggcctgcctggagggctgcttctgccccccagggctctacatggatgagaggggggactgcgtgcccaaggcccagtgcccctgttactatgacggtgagatcttccagccagaagacatcttctcagaccatcacaccatgtgctactgtgaggatggcttcatgcactgtaccatgagtggagtccccggaagcttgctgcctgacgctgtcctcagcagtcccctgtctcatcgcagcaaaaggagcctatcctgtcggccccccatggtcaagctggtgtgtcccgctgacaacctgcgggctgaagggctcgagtgtaccaaaacgtgccagaactatgacctggagtgcatgagcatgggctgtgtctctggctgcctctgccccccgggcatggtccggcatgagaacagatgtgtggccctggaaaggtgtccctgcttccatcagggcaaggagtatgcccctggagaaacagtgaagattggctgcaacacttgtgtctgtcgggaccggaagtggaactgcacagaccatgtgtgtgatgccacgtgctccacgatcggcatggcccactacctcaccttcgacgggctcaaatacctgttccccggggagtgccagtacgttctggtgcaggattactgcggcagtaaccctgggacctttcggatcctagtggggaataagggatgcagccacccctcagtgaaatgcaagaaacgggtcaccatcctggtggagggaggagagattgagctgtttgacggggaggtgaatgtgaagaggcccatgaaggatgagactcactttgaggtggtggagtctggccggtacatcattctgctgctgggcaaagccctctccgtggtctgggaccgccacctgagcatctccgtggtcctgaagcagacataccaggagaaagtgtgtggcctgtgtgggaattttgatggcatccagaacaatgacctcaccagcagcaacctccaagtggaggaagaccctgtggactttgggaactcctggaaagtgagctcgcagtgtgctgacaccagaaaagtgcctctggactcatcccctgccacctgccataacaacatcatgaagcagacgatggtggattcctcctgtagaatccttaccagtgacgtcttccaggactgcaacaagctggtggaccccgagccatatctggatgtctgcatttacgacacctgctcctgtgagtccattggggactgcgcctgcttctgcgacaccattgctgcctatgcccacgtgtgtgcccagcatggcaaggtggtgacctggaggacggccacattgtgcccccagagctgcgaggagaggaatctccgggagaacgggtatgagtgtgagtggcgctataacagctgtgcacctgcctgtcaagtcacgtgtcagcaccctgagccactggcctgccctgtgcagtgtgtggagggctgccatgcccactgccctccagggaaaatcctggatgagcttttgcagacctgcgttgaccctgaagactgtccagtgtgtgaggtggctggtcggcgttttgcctcaggaaagaaagtcaccttgaatcccagtgaccctgagcactgccagatttgtaaaacagattcctgggttgtttgaagtgatgaatcttattgcttctccaggggccagcttagagactaggttttgggtaaaaagtcctagccacatgagttgagagactgagtttattttatttgtttatttatttaattcattatttaatgtagtttattgttttgtttgttcctagggtttgcttctttttctttagggatggactagactgcttcctccacatatattcctccacagtgtttttgctcttaataggtttttaagtcctaacaggtttttgcttttctcactgaaggtgggggtatggtcacttattgtccttgtaggtcatgactagtctgagcacaggtgtccatatactggaggtggggactcaggagaggaaagtgagatgga

SEQ ID NO: 12 - DNA, 20 bp, Artificial Sequence
  CRISPR target site
caggtatttgagcccgtcga

SEQ ID NO: 13 - DNA, 20 bp, Artificial Sequence
  CRISPR target site
gctgggcaaagccctctccg

SEQ ID NO: 14 - DNA, 20 bp, Artificial Sequence
  CRISPR target site
tctttcctgaggcaaaacgc

SEQ ID NO: 15 - DNA, 4700 bp, Artificial Sequence
  Construct
gtgggcagagccattctcactaacttgccttgtgtgagctaagaccatttctttgccttgtgatttattccatccttatggcagttgggctgactccttgacgtgacaatatcaacagctgcgtgttggggatcacttgcagcatggatgttagtaagccagtagtattcagtactgtcacttggaaatgactgactgctcaaggacataggggatttttagtagaacttctggggaggctgcttagctctgatggtgcttccagacccatcatttgtcagtggttgtgctgcttgacagtaacccccatagggagaggttggctcaggcttgactatgagtgagggatgttagtggggcctccctgctccaccgaatctgtgtctcagctcttgggcatgtattcacctgccattgtagatgtggtcgtggtttgtctggaaagcttatctccaactttacttaggtccaagattagctttcaaatcatttatggtggtggtgccttcaatgtgaggatttttaccaaatcatcttatgtaggtgcaaaatgatcagtattcaccaatagtgttttaataggaatcttgagaatttgccaattatgtgaatgactcagggtcaaatgagggagcattgagactcaaccattttttttttttttttttttttggctctggaaatatttctcctgtgcgatctcttaagggattaagtcaacatggtcacttgtaccattaccttagtcacttggcagctgaaaggacacgtgtctttaggtgaattttctttacaatgagatcatgagctcatagcttgcttagcttttcttaatttccacgagtcataacacccctggaacttaactttgttttctgggccaaggcatccctgtagaatggattattcacactgtacatttaaattttttagaagtgtggttctataatttgccacattttatgtaacaggaaaatatttaatggccaagtgttacttacctaaacctctctacctctcagagccccagtttcctaatctgtaaaaaaaggaggaaattgttctatatgacctcaaagggcctgttccgttctctactgtatttatctgtgtgcaacttggtcacacctgcctgtctgcatgtagtaggcatgggggtttggataacgtcgcatccatcctctgcttctctctgtccaggcgtgtgcacaggcagcagtactcggcacatcgtgacctttgatgggcagaatttcaagctgactggcagctgttcttatgtcctatttcaaaacaaggagcaggacctggaggtgattctccataatggtgcctgcagccctggagcaaggcagggctgcatgaaatccatcgaggtgaagcacagtgccctctccgtcgagctgcacagtgacatggaggtgacggtgaatgggagactggtctctgttccttacgtgggtgggaacatggaagtcaacgtttatggtgccatcatgcatgaggtcagattcaatcaccttggtcacatcttcacattcactccacaaaacaatgagttccaactgcagctcagccccaagacttttgcttcaaagacgtatggtctgtgtgggatctgtgatgagaacggagccaatgacttcatgctgagggatggcacagtcaccacagactggaaaacacttgttcaggaatggactgtgcagcggccagggcagacgtgccagcccatcctggaggagcagtgtcttgtccccgacagctcccactgccaggtcctcctcttaccactgtttgctgaatgccacaaggtcctggctccagccacattctatgccatctgccagcaggacagttgccaccaggagcaagtgtgtgaggtgatcgcctcttatgcccacctctgtcggaccaacggggtctgcgttgactggaggacacctgatttctgtgctatgtcatgcccaccatctctggtctacaaccactgtgagcatggctgtccccggcactgtgatggcaacgtgagctcctgtggggaccatccctccgaaggctgtttctgccctccagataaagtcatgttggaaggcagctgtgtccctgaagaggcctgcactcagtgcattggtgaggatggagtccagcaccagttcctggaagcctgggtcccggaccaccagccctgtcagatctgcacatgcctcagcgggcggaaggtcaactgcacaacgcagccctgccccacggccaaagctcccacgtgtggcctgtgtgaagtagcccgcctccgccagaatgcagaccagtgctgccccgagtatgagtgtgtgtgtgacccagtgagctgtgacctgcccccagtgcctcactgtgaacgtggcctccagcccacactgaccaaccctggcgagtgcagacccaacttcacctgcgcctgcaggaaggaggagtgcaaaagagtgtccccaccctcctgccccccgcaccgtttgcccacccttcggaagacccagtgctgtgatgagtatgagtgtgcctgcaactgtgtcaactccacagtgagctgtccccttgggtacttggcctcaactgccaccaatgactgtggctgtaccacaaccacctgccttcccgacaaggtgtgtgtccaccgaagcaccatctaccctgtgggccagttctgggaggagggctgcgatgtgtgcacctgcaccgacatggaggatgccgtgatgggcctccgcgtggcccagtgctcccagaagccctgtgaggacagctgtcggtcgggcttcacttacgttctgcatgaaggcgagtgctgtggaaggtgcctgccatctgcctgtgaggtggtgactggctcaccgcggggggactcccagtcttcctggaagagtgtcggctcccagtgggcctccccggagaacccctgcctcatcaatgagtgtgtccgagtgaaggaggaggtctttatacaacaaaggaacgtctcctgcccccagctggaggtccctgtctgcccctcgggctttcagctgagctgtaagacctcagcgtgctgcccaagctgtcgctgtgagcgcatggaggcctgcatgctcaatggcactgtcattgggcccgggaagactgtgatgatcgatgtgtgcacgacctgccgctgcatggtgcaggtgggggtcatctctggattcaagctggagtgcaggaagaccacctgcaacccctgccccctgggttacaaggaagaaaataacacaggtgaatgttgtgggagatgtttgcctacggcttgcaccattcagctaagaggaggacagatcatgacactgaagcgtgatgagacgctccaggatggctgtgatactcacttctgcaaggtcaatgagagaggagagtacttctgggagaagagggtcacaggctgcccaccctttgatgaacacaagtgtctggctgagggaggtaaaattatgaaaattccaggcacctgctgtgacacatgtgaggagcctgagtgcaacgacatcactgccaggctgcagtatgtcaaggtgggaagctgtaagtctgaagtagaggtggatatccactactgccagggcaaatgtgccagcaaagccatgtactccattgacatcaacgatgtgcaggaccagtgctcctgctgctctccgacacggacggagcccatgcaggtggccctgcactgcaccaatggctctgttgtgtaccatgaggttctcaatgccatggagtgcaaatgctcccccaggaagtgcagcaagtgactgactgaaacttgtttattgcagcttataatggttacaaataaagcaatagcatcacaaatttcacaaataaagcatttttttcactgcattctagttgtggtttgtccaaactcatcaatgtatcttatcatgtctggatcgtgagaagtactttctgtggatccgtggtaaggcaatagaatgtcaggaaaaccacctggacctggtggcagttgcttttagttgatgctcttgttaggagctctgccttctgcttaagtggaggagaggagtaccactttcttagaggggtttattgccatccccttgtcttggcgtgatttcatgttgttccgggctcagatttgcaagatggaatcacttttagatagcataaaattgtgaatttagtgccagtttctggcactggtggagaattgggattggcatcaggattgtttactcggaaggtattatgagtccaatgcctaaaccctgtaagctttccaaagggaaacatttatggcctaaattaggtcttttgaaaatatttaaggcctacataaaacgtcaggctccaaaatttgaaaagaaaactgcaaaactgatatatatatatataaatgattgattaaatgcttacaaaaggttacactatgccaacttctttacttgttcgtgtagaaatcataaatatttcattgtaatgtgaaaacaacttgtaagctagattttctcacttcgcaagaattcctgaatttgaacaataattgcagaaaaatcttagtcatatatcaagtgagtaactcatagccaaaaattaaaaaatcaaaatgataaaaaatccttccaaaaattttacagcaaaattatattcattgtggaatgtgagtacattttaatgtgtttgatatgata

SEQ ID NO: 16 - DNA, 4700 bp, Artificial Sequence
  Construct
cttccccatgggcctagactaactctcaacatgggtataaagggctttagaaatacgataacacggagactcatatcaaagtaccatagtttaagttgattttaggttagaaacttaaaaaatatgcttttggccaggtgcagtggctcacgtctgtaatcccagcactttgggaggccgaggcgggcggatcacgaggtcaggagatcgagaccatcttggctaacacagtgaaaccctgtctctactaaaaatacaaaaaattagccgggcgtggtggcgggcacctgtagtaccagctacttgggaggctgaggcaggagaatggtgtgaacccgggaggcagagcttgcagtgagccgagatcgcgccactgtactccagcctgggcaacagagtgagactccatctcaaaaaaaaaaaaaaaatacacacacacacacacacacgctttttttgtggcgggggcctgggtttgtatattttcccgttactagatgtaagtcaaaacctgcataaagctactgtccttcgggggaataagtcaatgcaagtttgcccttaaagggcaataactctatgcaagttttgacttatagctaataacattagctgtacagagagatggcagctctcctggtaggaatcttcaagtagatctctttcaggtttccaggatcttgcttcatctccccaccttccccatccctggcgtgatctacatgtgaaccaagataatgacagcgtaagctgtagttattgccatattatcgctgttgttggcatcataattattaataactgcagagcatgtctgaagaaccacaggatgaccacctcagcctcatgtccctatgtctccactgttaaccttgttcagattcttttcagagttgagttgacttcaaaaactagaccaggttgcttaagcagacattgtgaatggttcagaatttctgggtgaaagatgggaactaaggtcttatttgtgtctgttgcaggatttgttaggatttgcatggatgaggatggtaatgagaagaggcccggggacgtctggaccttgccagaccagtgccacaccgtgacttgccagccagatggccagaccttgctgaagagtcatcgggtcaactgtgaccgggggctgaggccttcgtgccctaacagccagtcccctgttaaagtggaagagacctgtggctgccgctggacctgcccctgcgtgtgcacaggcagctccactcggcacatcgtgacctttgatgggcagaatttcaagctgactggcagctgttcttatgtcctatttcaaaacaaggagcaggacctggaggtgattctccataatggtgcctgcagccctggagcaaggcagggctgcatgaaatccatcgaggtgaagcacagtgccctctccgtcgagctgcacagtgacatggaggtgacggtgaatgggagactggtctctgttccttacgtgggtgggaacatggaagtcaacgtttatggtgccatcatgcatgaggtcagattcaatcaccttggtcacatcttcacattcactccacaaaacaatgagttccaactgcagctcagccccaagacttttgcttcaaagacgtatggtctgtgtgggatctgtgatgagaacggagccaatgacttcatgctgagggatggcacagtcaccacagactggaaaacacttgttcaggaatggactgtgcagcggccagggcagacgtgccagcccatcctggaggagcagtgtcttgtccccgacagctcccactgccaggtcctcctcttaccactgtttgctgaatgccacaaggtcctggctccagccacattctatgccatctgccagcaggacagttgccaccaggagcaagtgtgtgaggtgatcgcctcttatgcccacctctgtcggaccaacggggtctgcgttgactggaggacacctgatttctgtgctatgtcatgcccaccatctctggtctacaaccactgtgagcatggctgtccccggcactgtgatggcaacgtgagctcctgtggggaccatccctccgaaggctgtttctgccctccagataaagtcatgttggaaggcagctgtgtccctgaagaggcctgcactcagtgcattggtgaggatggagtccagcaccagttcctggaagcctgggtcccggaccaccagccctgtcagatctgcacatgcctcagcgggcggaaggtcaactgcacaacgcagccctgccccacggccaaagctcccacgtgtggcctgtgtgaagtagcccgcctccgccagaatgcagaccagtgctgccccgagtatgagtgtgtgtgtgacccagtgagctgtgacctgcccccagtgcctcactgtgaacgtggcctccagcccacactgaccaaccctggcgagtgcagacccaacttcacctgcgcctgcaggaaggaggagtgcaaaagagtgtccccaccctcctgccccccgcaccgtttgcccacccttcggaagacccagtgctgtgatgagtatgagtgtgcctgcaactgtgtcaactccacagtgagctgtccccttgggtacttggcctcaactgccaccaatgactgtggctgtaccacaaccacctgccttcccgacaaggtgtgtgtccaccgaagcaccatctaccctgtgggccagttctgggaggagggctgcgatgtgtgcacctgcaccgacatggaggatgccgtgatgggcctccgcgtggcccagtgctcccagaagccctgtgaggacagctgtcggtcgggcttcacttacgttctgcatgaaggcgagtgctgtggaaggtgcctgccatctgcctgtgaggtggtgactggctcaccgcggggggactcccagtcttcctggaagagtgtcggctcccagtgggcctccccggagaacccctgcctcatcaatgagtgtgtccgagtgaaggaggaggtctttatacaacaaaggaacgtctcctgcccccagctggaggtccctgtctgcccctcgggctttcagctgagctgtaagacctcagcgtgctgcccaagctgtcgctgtgagcgcatggaggcctgcatgctcaatggcactgtcattgggcccgggaagactgtgatgatcgatgtgtgcacgacctgccgctgcatggtgcaggtgggggtcatctctggattcaagctggagtgcaggaagaccacctgcaacccctgccccctgggttacaaggaagaaaataacacaggtgaatgttgtgggagatgtttgcctacggcttgcaccattcagctaagaggaggacagatcatgacactgaagcgtgatgagacgctccaggatggctgtgatactcacttctgcaaggtcaatgagagaggagagtacttctgggagaagagggtcacaggctgcccaccctttgatgaacacaagtgtctggctgagggaggtaaaattatgaaaattccaggcacctgctgtgacacatgtgaggagcctgagtgcaacgacatcactgccaggctgcagtatgtcaaggtgggaagctgtaagtctgaagtagaggtggatatccactactgccagggcaaatgtgccagcaaagccatgtactccattgacatcaacgatgtgcaggaccagtgctcctgctgctctccgacacggacggagcccatgcaggtggccctgcactgcaccaatggctctgttgtgtaccatgaggttctcaatgccatggagtgcaaatgctcccccaggaagtgcagcaagtgactgactgaaacttgtttattgcagcttataatggttacaaataaagcaatagcatcacaaatttcacaaataaagcatttttttcactgcattctagttgtggtttgtccaaactcatcaatgtatcttatcatgtctggatcgtaagttcctttctgttgactttgaaagaaaggttagagatgtgtttggggctcttgttcccactggttaatttttcctcctttggtcttagtccagtgcttccttttactattatcttgtttttgcgggtccatctgtacatcttgtgttttgcttcctgtctcatgtacagggggcctccttgctgtgtaggcctgtgttcaattctaggggtcagttgtctggcagatgggcttagagttggagtacctcatcttattccctgcctgaatctgctgttttcttctgcagcccggggacgtctggaccttgccagaccagtgccacaccgtgacttgccagccagatggccagaccttgctgaagagtcatcgggtcaactgtgaccgggggctgaggccttcgtgccctaacagccagtcccctgttaaagtggaagagacctgtggctgccgctggacctgcccctgtgagtcctttgcttctccagccagggcagcgtcaaaggggcagtgcttttagcttggctgggcagaaaagtagagcaggcacccaccagcccagaagtaccctttccctcatcaccacatgcacagtgctaccttcactcaccttcctttcctcctgtgctctttggacatgcatgcagccagtctcagggatcactgccctctttctctgtctttggaaggcacttccccagattatgcataactggaaggaagaattgctt

SEQ ID NO: 17 - DNA, 4900 bp, Artificial Sequence
  Construct
acaaaggataaggcaaatacttaacctctttgtatatcatctatacaaaggatacacatctttgtgtatcctcatctataaaatggggataataatagcacctacttgcttaaaatagtatctggcacatgacaagtgctcaaaaaaaaatgcttgctcccactgctgttactactaccttttactgacactggcgtctaatccattcctagttcctgaacatctttattcggtgtgtttgggaccatcccagaataataagccttcttttagtatcttttgagtcacacacttgtcagtacttttgtttctttgtttttgtttttattcacggccatagatttatttaaattcttgtaatatttctgctgaggaaaacaacaattacatcatttcatcaaatctcagatgtgctcagacactaacaggagcactaggcatttatagctggaagaatcacagtatgttcacctgccctgcaagatctgaggacacagcagctcattgtccagggaggggctgccgctccatttccttttgcagtctccttgtattgccaggccagtattttactcatttctagaagaatggtggccccttcttaccgaggaagcctatgcctgctgcttttatttgtagacatttaaacttcctttgggtagaattggagtcttctcagtgtctctaaatctgaggtagtccggacccaggaactccatctccccatcccctcctccctggcccacattgcccttgtactcacgaaggcaccccccgccccccttggtggtgccacgtggtcagcacgccctgcagatcctattggatgtcaggttgtaggcctggtggccattgtccctgctggcacctgtgtgctcaccttcctggttgtctttgcagactgcagccagcccctggacgtgatccttctcctggatggctcctccagtttcccagcttcttattttgatgaaatgaagagtttcgcaaaagctttcatttcaaaagccaatatagggcctcgtctcactcaggtgtcagtgctgcagtatggaagcatcaccaccattgacgtgccatggaacgtggtcccggagaaagcccatttgctgagccttgtggacgtcatgcagcgggagggaggccccagccaaatcggggatgccttgggctttgctgtgcgatacttgacttcagaaatgcatggtgccaggccgggagcctcaaaggcggtggtcatcctggtcacggacgtctctgtggattcagtggatgcagcagctgatgccgccaggtccaacagagtgacagtgttccctattggaattggagatcgctacgatgcagcccagctacggatcttggcaggcccagcaggcgactccaacgtggtgaagctccagcgaatcgaagacctccctaccatggtcaccttgggcaattccttcctccacaaactgtgctctggatttgttaggatttgcatggatgaggatgggaatgagaagaggcccggggacgtctggaccttgccagaccagtgccacaccgtgacttgccagccagatggccagaccttgctgaagagtcatcgggtcaactgtgaccgggggctgaggccttcgtgccctaacagccagtcccctgttaaagtggaagagacctgtggctgccgctggacctgcccctgcgtgtgcacaggcagctccactcggcacatcgtgacctttgatgggcagaatttcaagctgactggcagctgttcttatgtcctatttcaaaacaaggagcaggacctggaggtgattctccataatggtgcctgcagccctggagcaaggcagggctgcatgaaatccatcgaggtgaagcacagtgccctctccgtcgagctgcacagtgacatggaggtgacggtgaatgggagactggtctctgttccttacgtgggtgggaacatggaagtcaacgtttatggtgccatcatgcatgaggtcagattcaatcaccttggtcacatcttcacattcactccacaaaacaatgagttccaactgcagctcagccccaagacttttgcttcaaagacgtatggtctgtgtgggatctgtgatgagaacggagccaatgacttcatgctgagggatggcacagtcaccacagactggaaaacacttgttcaggaatggactgtgcagcggccagggcagacgtgccagcccatcctggaggagcagtgtcttgtccccgacagctcccactgccaggtcctcctcttaccactgtttgctgaatgccacaaggtcctggctccagccacattctatgccatctgccagcaggacagttgccaccaggagcaagtgtgtgaggtgatcgcctcttatgcccacctctgtcggaccaacggggtctgcgttgactggaggacacctgatttctgtgctatgtcatgcccaccatctctggtctacaaccactgtgagcatggctgtccccggcactgtgatggcaacgtgagctcctgtggggaccatccctccgaaggctgtttctgccctccagataaagtcatgttggaaggcagctgtgtccctgaagaggcctgcactcagtgcattggtgaggatggagtccagcaccagttcctggaagcctgggtcccggaccaccagccctgtcagatctgcacatgcctcagcgggcggaaggtcaactgcacaacgcagccctgccccacggccaaagctcccacgtgtggcctgtgtgaagtagcccgcctccgccagaatgcagaccagtgctgccccgagtatgagtgtgtgtgtgacccagtgagctgtgacctgcccccagtgcctcactgtgaacgtggcctccagcccacactgaccaaccctggcgagtgcagacccaacttcacctgcgcctgcaggaaggaggagtgcaaaagagtgtccccaccctcctgccccccgcaccgtttgcccacccttcggaagacccagtgctgtgatgagtatgagtgtgcctgcaactgtgtcaactccacagtgagctgtccccttgggtacttggcctcaactgccaccaatgactgtggctgtaccacaaccacctgccttcccgacaaggtgtgtgtccaccgaagcaccatctaccctgtgggccagttctgggaggagggctgcgatgtgtgcacctgcaccgacatggaggatgccgtgatgggcctccgcgtggcccagtgctcccagaagccctgtgaggacagctgtcggtcgggcttcacttacgttctgcatgaaggcgagtgctgtggaaggtgcctgccatctgcctgtgaggtggtgactggctcaccgcggggggactcccagtcttcctggaagagtgtcggctcccagtgggcctccccggagaacccctgcctcatcaatgagtgtgtccgagtgaaggaggaggtctttatacaacaaaggaacgtctcctgcccccagctggaggtccctgtctgcccctcgggctttcagctgagctgtaagacctcagcgtgctgcccaagctgtcgctgtgagcgcatggaggcctgcatgctcaatggcactgtcattgggcccgggaagactgtgatgatcgatgtgtgcacgacctgccgctgcatggtgcaggtgggggtcatctctggattcaagctggagtgcaggaagaccacctgcaacccctgccccctgggttacaaggaagaaaataacacaggtgaatgttgtgggagatgtttgcctacggcttgcaccattcagctaagaggaggacagatcatgacactgaagcgtgatgagacgctccaggatggctgtgatactcacttctgcaaggtcaatgagagaggagagtacttctgggagaagagggtcacaggctgcccaccctttgatgaacacaagtgtctggctgagggaggtaaaattatgaaaattccaggcacctgctgtgacacatgtgaggagcctgagtgcaacgacatcactgccaggctgcagtatgtcaaggtgggaagctgtaagtctgaagtagaggtggatatccactactgccagggcaaatgtgccagcaaagccatgtactccattgacatcaacgatgtgcaggaccagtgctcctgctgctctccgacacggacggagcccatgcaggtggccctgcactgcaccaatggctctgttgtgtaccatgaggttctcaatgccatggagtgcaaatgctcccccaggaagtgcagcaagtgactgactgaaacttgtttattgcagcttataatggttacaaataaagcaatagcatcacaaatttcacaaataaagcatttttttcactgcattctagttgtggtttgtccaaactcatcaatgtatcttatcatgtctggatcgtgggtgagcgaggcacctgaagcagcaggtgacgaagaggctctttttgtggctctacttgattcaaaataatccgcattttctcgttccgtttagggcctcgtctcactcaggtgtcagtgctgcagtatggaagcatcaccaccattgacgtgccatggaacgtggtcccggagaaagcccatttgctgagccttgtggacgtcatgcagcgggagggaggccccagccaaatcggtaacgttggtgccacaggctggatgcagaagctgcattctggttcttatttttggcataagtgactgtgtgacctcggccagtcactttgctccttggccttagtttcttctcctggaaagtgaggggctagatgctcttccacgtctctccagatctcaactgggtgttccttggagtttctgaatcattcagcttttaagtgacttaaggatccaccgttaagacag

SEQ ID NO: 18 - DNA, 20 bp, Artificial Sequence
  CRISPR target site
aaaggtcacgatgtgccgag

SEQ ID NO: 19 - DNA, 20 bp, Artificial Sequence
  CRISPR target site
ggatttgcatggatgaggat

SEQ ID NO: 20 - DNA, 20 bp, Artificial Sequence
  CRISPR target site
tgaaatgaagagtttcgcca

SEQ ID NO: 21 - protein, 639 aa, Scytonema hoffmanni
MSQITIQARLISFESNRQQLWKLMADLNTPLINELLCQLGQHPDFEKWQQKGKLPSTVVSQLCQPLKTDPRFAGQPSRLYMSAIHIVDYIYKSWLAIQKRLQQQLDGKTRWLEMLNSDAELVELSGDTLEAIRVKAAEILAIAMPASESDSASPKGKKGKKEKKPSSSSPKRSLSKTLFDAYQETEDIKSRSAISYLLKNGCKLTDKEEDSEKFAKRRRQVEIQIQRLTEKLISRMPKGRDLTNAKWLETLLTATTTVAEDNAQAKRWQDILLTRSSSLPFPLVFETNEDMVWSKNQKGRLCVHFNGLSDLIFEVYCGNRQLHWFQRFLEDQQTKRKSKNQHSSGLFTLRNGHLVWLEGEGKGEPWNLHHLTLYCCVDNRLWTEEGTEIVRQEKADEITKFITNMKKKSDLSDTQQALIQRKQSTLTRINNSFERPSQPLYQGQSHILVGVSLGLEKPATVAVVDAIANKVLAYRSIKQLLGDNYELLNRQRRQQQYLSHERHKAQKNFSPNQFGASELGQHIDRLLAKAIVALARTYKAGSIVLPKLGDMREVVQSEIQAIAEQKFPGYIEGQQKYAKQYRVNVHRWSYGRLIQSIQSKAAQTGIVIEEGKQPIRGSPHDKAKELALSAYNLRLTRRS

SEQ ID NO: 22 - protein, 642 aa, Anabaena cylindrica
MSVITIQCRLVAEEDSLRQLWELMSEKNTPFINEILLQIGKHPEFETWLEKGRIPAELLKTLGNSLKTQEPFTGQPGRFYTSAITLVDYLYKSWFALQKRRKQQIEGKQRWLKMLKSDQELEQESQSSLEVIRNKATELFSKFTPQSDSEALRRNQNDKQKKVKKTKKSTKPKTSSIFKIFLSTYEEAEEPLTRCALAYLLKNNCQISELDENPEEFTRNKRRKEIEIERLKDQLQSRIPKGRDLTGEEWLETLEIATFNVPQNENEAKAWQAALLRKTANVPFPVAYESNEDMTWLKNDKNRLFVRFNGLGKLTFEIYCDKRHLHYFQRFLEDQEILRNSKRQHSSSLFTLRSGRIAWLPGEEKGEHWKVNQLNFYCSLDTRMLTTEGTQQVVEEKVTAITEILNKTKQKDDLNDKQQAFITRQQSTLARINNPFPRPSKPNYQGKSSILIGVSFGLEKPVTVAVVDVVKNKVIAYRSVKQLLGENYNLLNRQRQQQQRLSHERHKAQKQNAPNSFGESELGQYVDRLLADAIIAIAKKYQAGSIVLPKLRDMREQISSEIQSRAENQCPGYKEGQQKYAKEYRINVHRWSYGRLIESIKSQAAQAGIAIETGKQSIRGSPQEKARDLAVFTYQERQAALI

SEQ ID NO: 23 - DNA, 208 bp, Artificial Sequence
  Left end for ShCas12k
tacagtgacaaattatctgtcgtcggtgacagattaatgtcattgtgactatttaattgtcgtcgtgacccatcagcgttgcttaattaattgatgacaaattaaatgtcatcaatataatatgctctgcaattattatacaaagcaattaaaacaagcggataaaaggacttgctttcaacccacccctaagtttaatagttactga

SEQ ID NO: 24 - DNA, 219 bp, Artificial Sequence
  Right end for ShCas12k
cgacagtcaatttgtcattatgaaaatacacaaaagctttttcctatcttgcaaagcgacagctaatttgtcacaatcacggacaacgacatctattttgtcactgcaaagaggttatgctaaaactgccaaagcgctataatctatactgtataaggattttactgatgacaataatttgtcacaacgacatataattagtcactgtacacgtagaga

SEQ ID NO: 25 - DNA, 23 bp, Artificial Sequence
  Synthetic sequence
tgtatttctgttcagggagatgg

SEQ ID NO: 26 - DNA, 23 bp, Artificial Sequence
  Synthetic sequence
agatgtactgccaagtaggaaag

SEQ ID NO: 27 - DNA, 20 bp, Artificial Sequence
  Synthetic sequence
ccatcacaccatgtgctact

SEQ ID NO: 28 - DNA, 20 bp, Artificial Sequence
  Synthetic sequence
tccattcagaccacaccaag

SEQ ID NO: 29 - DNA, 20 bp, Artificial Sequence
  Synthetic sequence
gggatgggaggtgaattctt

SEQ ID NO: 30 - DNA, 20 bp, Artificial Sequence
  Synthetic sequence
gggatgggaggtgaattctt

SEQ ID NO: 31 - DNA, 20 bp, Artificial Sequence
  Synthetic sequence
acgttctggtgcaggattac

SEQ ID NO: 32 - DNA, 22 bp, Artificial Sequence
  Synthetic sequence
tggcccatgactcaatgataag

SEQ ID NO: 33 - DNA, 22 bp, Artificial Sequence
  Synthetic sequence
ccgatagaactttctgcagtgg

SEQ ID NO: 34 - DNA, 23 bp, Artificial Sequence
  Synthetic sequence
ctgtagaatccttaccagtgacg

SEQ ID NO: 35 - DNA, 19 bp, Artificial Sequence
  Synthetic sequence
cctgccaccttgactatgg

SEQ ID NO: 36 - DNA, 22 bp, Artificial Sequence
  Synthetic sequence
tatgcagaggagataggagagg

SEQ ID NO: 37 - DNA, 20 bp, Artificial Sequence
  Synthetic sequence
gatcccacacagaccatacg

SEQ ID NO: 38 - DNA, 22 bp, Artificial Sequence
  Synthetic sequence
gcattctagttgtggtttgtcc

SEQ ID NO: 39 - DNA, 21 bp, Artificial Sequence
  Synthetic sequence
gtgtctccaagagcatctagc

SEQ ID NO: 40 - DNA, 21 bp, Artificial Sequence
  Synthetic sequence
gtgcccatgcataagatttgg

SEQ ID NO: 41 - DNA, 21 bp, Artificial Sequence
  Synthetic sequence
ccagtcagcttgaaattctgc

SEQ ID NO: 42 - DNA, 24 bp, Artificial Sequence
  Synthetic sequence
tgttcagcataaaggttacaatcc

SEQ ID NO: 43 - DNA, 20 bp, Artificial Sequence
  Synthetic sequence
gatgtcaggtgtcaggtagc

SEQ ID NO: 44 - DNA, 22 bp, Artificial Sequence
  Synthetic sequence
atgatcactcctggacacaaag

SEQ ID NO: 45 - DNA, 23 bp, Artificial Sequence
  CAST target site
gggctgggaagtcagtcccgctc

SEQ ID NO: 46 - DNA, 23 bp, Artificial Sequence
  CAST target site
gaattgatccctttaccattatg

SEQ ID NO: 47 - DNA, 23 bp, Artificial Sequence
  CAST target site
tgaagtgatgaatcttattgctt

SEQ ID NO: 48 - protein, 2813 aa, Homo sapiens
MIPARFAGVLLALALILPGTLCAEGTRGRSSTARCSLFGSDFVNTFDGSMYSFAGYCSYLLAGGCQKRSFSIIGDFQNGKRVSLSVYLGEFFDIHLFVNGTVTQGDQRVSMPYASKGLYLETEAGYYKLSGEAYGFVARIDGSGNFQVLLSDRYFNKTCGLCGNFNIFAEDDFMTQEGTLTSDPYDFANSWALSSGEQWCERASPPSSSCNISSGEMQKGLWEQCQLLKSTSVFARCHPLVDPEPFVALCEKTLCECAGGLECACPALLEYARTCAQEGMVLYGWTDHSACSPVCPAGMEYRQCVSPCARTCQSLHINEMCQERCVDGCSCPEGQLLDEGLCVESTECPCVHSGKRYPPGTSLSRDCNTCICRNSQWICSNEECPGECLVTGQSHFKSFDNRYFTFSGICQYLLARDCQDHSFSIVIETVQCADDRDAVCTRSVTVRLPGLHNSLVKLKHGAGVAMDGQDVQLPLLKGDLRIQHTVTASVRLSYGEDLQMDWDGRGRLLVKLSPVYAGKTCGLCGNYNGNQGDDFLTPSGLAEPRVEDFGNAWKLHGDCQDLQKQHSDPCALNPRMTRFSEEACAVLTSPTFEACHRAVSPLPYLRNCRYDVCSCSDGRECLCGALASYAAACAGRGVRVAWREPGRCELNCPKGQVYLQCGTPCNLTCRSLSYPDEECNEACLEGCFCPPGLYMDERGDCVPKAQCPCYYDGEIFQPEDIFSDHHTMCYCEDGFMHCTMSGVPGSLLPDAVLSSPLSHRSKRSLSCRPPMVKLVCPADNLRAEGLECTKTCQNYDLECMSMGCVSGCLCPPGMVRHENRCVALERCPCFHQGKEYAPGETVKIGCNTCVCQDRKWNCTDHVCDATCSTIGMAHYLTFDGLKYLFPGECQYVLVQDYCGSNPGTFRILVGNKGCSHPSVKCKKRVTILVEGGEIELFDGEVNVKRPMKDETHFEVVESGRYIILLLGKALSVVWDRHLSISVVLKQTYQEKVCGLCGNFDGIQNNDLTSSNLQVEEDPVDFGNSWKVSSQCADTRKVPLDSSPATCHNNIMKQTMVDSSCRILTSDVFQDCNKLVDPEPYLDVCIYDTCSCESIGDCACFCDTIAAYAHVCAQHGKVVTWRTATLCPQSCEERNLRENGYECEWRYNSCAPACQVTCQHPEPLACPVQCVEGCHAHCPPGKILDELLQTCVDPEDCPVCEVAGRRFASGKKVTLNPSDPEHCQICHCDVVNLTCEACQEPGGLVVPPTDAPVSPTTLYVEDISEPPLHDFYCSRLLDLVFLLDGSSRLSEAEFEVLKAFVVDMMERLRISQKWVRVAVVEYHDGSHAYIGLKDRKRPSELRRIASQVKYAGSQVASTSEVLKYTLFQIFSKIDRPEASRITLLLMASQEPQRMSRNFVRYVQGLKKKKVIVIPVGIGPHANLKQIRLIEKQAPENKAFVLSSVDELEQQRDEIVSYLCDLAPEAPPPTLPPDMAQVTVGPGLLGVSTLGPKRNSMVLDVAFVLEGSDKIGEADFNRSKEFMEEVIQRMDVGQDSIHVTVLQYSYMVTVEYPFSEAQSKGDILQRVREIRYQGGNRTNTGLALRYLSDHSFLVSQGDREQAPNLVYMVTGNPASDEIKRLPGDIQVVPIGVGPNANVQELERIGWPNAPILIQDFETLPREAPDLVLQRCCSGEGLQIPTLSPAPDCSQPLDVILLLDGSSSFPASYFDEMKSFAKAFISKANIGPRLTQVSVLQYGSITTIDVPWNVVPEKAHLLSLVDVMQREGGPSQIGDALGFAVRYLTSEMHGARPGASKAVVILVTDVSVDSVDAAADAARSNRVTVFPIGIGDRYDAAQLRILAGPAGDSNVVKLQRIEDLPTMVTLGNSFLHKLCSGFVRICMDEDGNEKRPGDVWTLPDQCHTVTCQPDGQTLLKSHRVNCDRGLRPSCPNSQSPVKVEETCGCRWTCPCVCTGSSTRHIVTFDGQNFKLTGSCSYVLFQNKEQDLEVILHNGACSPGARQGCMKSIEVKHSALSVELHSDMEVTVNGRLVSVPYVGGNMEVNVYGAIMHEVRFNHLGHIFTFTPQNNEFQLQLSPKTFASKTYGLCGICDENGANDFMLRDGTVTTDWKTLVQEWTVQRPGQTCQPILEEQCLVPDSSHCQVLLLPLFAECHKVLAPATFYAICQQDSCHQEQVCEVIASYAHLCRTNGVCVDWRTPDFCAMSCPPSLVYNHCEHGCPRHCDGNVSSCGDHPSEGCFCPPDKVMLEGSCVPEEACTQCIGEDGVQHQFLEAWVPDHQPCQICTCLSGRKVNCTTQPCPTAKAPTCGLCEVARLRQNADQCCPEYECVCDPVSCDLPPVPHCERGLQPTLTNPGECRPNFTCACRKEECKRVSPPSCPPHRLPTLRKTQCCDEYECACNCVNSTVSCPLGYLASTATNDCGCTTTTCLPDKVCVHRSTIYPVGQFWEEGCDVCTCTDMEDAVMGLRVAQCSQKPCEDSCRSGFTYVLHEGECCGRCLPSACEVVTGSPRGDSQSSWKSVGSQWASPENPCLINECVRVKEEVFIQQRNVSCPQLEVPVCPSGFQLSCKTSACCPSCRCERMEACMLNGTVIGPGKTVMIDVCTTCRCMVQVGVISGFKLECRKTTCNPCPLGYKEENNTGECCGRCLPTACTIQLRGGQIMTLKRDETLQDGCDTHFCKVNERGEYFWEKRVTGCPPFDEHKCLAEGGKIMKIPGTCCDTCEEPECNDITARLQYVKVGSCKSEVEVDIHYCQGKCASKAMYSIDINDVQDQCSCCSPTRTEPMQVALHCTNGSVVYHEVLNAMECKCSPRKCSK